SEQUENCE LISTING (28 sequences)

SEQ ID NO: 1 - DNA, 782 bp, Spodoptera frugiperda
  source 1..782: mol_type = other DNA
gatcgtcaagctcaatggaacaaacaggctcgaagctttgaatacgaagtctgcagcagccctggcggtgaaagcctccgtctttgtgactctcttcctgactctaataacatttaccagttgtgacacgggcaccataaaaggattgcagacaggctactatgaacaaccctcgtgcagcgctgcttgtggatgtacctccgagcgctacggcttccacccagtctgtatgctggagacgagggaaacttacttctccccgtgccacgctgggtgcaaccgaatgggggacctcaacggaatctcaatgttctacgactgtggatgcggcgcaggcgcacagaatgctgtgcgtggctcgtgcatgttggagaactgtctcgccaagtacgccaccttccagatccagttcgtgctcatgttggcagtcgcaggagctggtttcataatgcaaggtatggtgatactccggtcggtgcgaccagcagacaaggtgaccgccatgggagtcagcatgtctgtggtctcactcctcgccttcgtgttgggacattttgtctacatgactgtcagcaaaatgacatgtgtgtacgtctcgaacggcgaatgtctgctgcacagcacttcgatctggttcctgagtgccatcagcgtggggctggcagtactgtccggagcaggaagctacatcgtcagcaaactgagaactgacacggatgataacgagatctgtcaattataatctttattgcagaatattttttgtactgagagaccaaggt

SEQ ID NO: 2 - DNA, 1950 bp, Spodoptera frugiperda
  source 1..1950: mol_type = other DNA
atggtggtaaatggcttcgtgaacgtggtgatcaccaccattgagaagaggttcgggttacggtccatgcagactggagtcatagctggaggctacgacatggcatcgttcgcatgtctggccccagtcacctacttgggaggtcgctcagggtcctccaagcctcgctggttggggctaggagtactcctcatgggcacagggtctctactctttgccctgccacacttcctcgtgcctaactacaagatagctgggaatgaacctgctgatctatgcactcctaataggacggtaagagatagtatttattttcagttgacagacagttgcgatggtaacgtgccgtcggagggcggcgcgtgggcagtcgcagtgttcgtgttcgcgcaactgttgcacggcgcgggcgcaacaccgctgttcacgctcggcgtaacttacattgatgagaacgtgtccaagaagatgtcttctgtttacttgggtgtatactacacgatggcggtggtgggcccagcgatgggctatgtgatcggtggacaaatgctcaccatctacacagacttcctcactgtcgactccgacgcgttaggcgtaactccagtcagctccgtttggataggcgcctggtggatagggttcatactatccgccattttgtgtctcatcgtggccataccattgatggccttccctcatgaactgcctggtgcagctgagattagagcgtcgaaggtatctgaagctcacgaaggagctgcctccaagtcagcagcctttagcgccttacacgagctgcctcgtgccgccgccgccttgctccggaaccccaccttcctgttcctcaacctggcaggtgctactgaaggcatgctcatatcagggttcgctgcgttcttgccaaagttgattgaaaaccagtttgctgtcaacgcttcagaagctgctttacttttgggagtgatcacagtaccagcgggtggtggtggcacattcctcggagggtggctggtgaagcgctggcagctggcgtgcgctggcatcatcaagctgtgcgtggcagccaccgtggtggccgctgccttcaccttcggcttcgtgctcacttgtgacgactacccctttgctggagtcaccgtcatgtacaatagccctgcagtgccaggtgccgacagtctgacagcaatgtgcaacacggactgctcgtgtatcaacgctccgtactcgcccgtgtgtggcgccgatggcaacgtgtactactccgcctgccacgctggctgcaccaggcagaccctcgcgggcgccgcgcagctcttccaccagtgctcctgtgtactcaccaaagaaaatatgacgctaccgacatatgaacttgaaggagaagtcaactcgacgatctcttacgaagccatcaatagcatctgttcaacggactgcccattactttgggtgttcgtgtgcatgtcgtttggagtcatgctgttcacgttcctcgctaccatgcctgcgttgtcagctacattgaggtgtgtgcgagaagaccagcgttcgttcgcattgggcatccagtggatcaaagtccgtctgctgggtacgatcccggcgcctctgctgtttgggttcctcatcgacctgtcatgctccctgtggtctacctcttgtaacaagacgggtgcctgcttgcagtatgacaatactaatatgagcaggtacatgctagggattgcattggtgggcaagctctgttctttgctattcttcttcctggcgtggtggttctaccgtccaccaggcagcaagaacggcaacgccatagttccctcagtagatctagtggtctgcgccgagaagaccaatgggaacgtcacgaatgtggccaatgggacgttggagagaggcagcaatggctactgtaatgcagcgttagaatgtagtgaccatttgtaa

SEQ ID NO: 3 - DNA, 2080 bp, Spodoptera frugiperda
  source 1..2080: mol_type = other DNA
attgtttcaggtagcggtgcgttaaatgagtttgacttctccctgacacctctgctaacatgttccggtttcttcatcatgtatctggtggaagaactggtgcacatgtacattcactacagagagagaaagaacggccaagtggcgccactcgtcaggaacctgagtgtccgtcagagccgcagcggagtcagcaatggagagaagagcgtgaataactcgaccgctgaccttactgagccaaaaaaggacattgagagaaatcaccacaatggacacggacattcacaccaccacagccacatgccaatgaacgtgggtgatgacgtaacttctgctctgagaggattacttattgtgcttgctctgtctatccacgaactgttcgagggcttggcagtcggtttagaatccagcacttctcacgtctggtacatgttaggagctgtttcagcccacaagctcgtcattgccttctgtattggtgtggagctgatcgcaacgcgtaccaagacttggttgatgattgtgtacatcaccactttcgccattgtttcgcccttgggtatcggtattggtctgctgttggtgggaggtcaaggcgctgccgctgccggtatctcgtccgtggttctccagggtctggcatcaggaactctgctctacgtcatcttctttgaaatctggaaaggcgacagaaccggtatcctgcagttcatcgcttctgtagtcggattcttcattatgttcggtctacagctgttaactggtcactcacactcgcactcccacggcggacacgaccacggccacagccacgacaggaaataagtacaatgagtccaatgacccacagcacgccaccattttctacgagaatataaaatgtgaatcgagtacttaatctaaacaagtgttgaccaaatgacaataattctatttgtgataaattattaagtgtaaatgttaattataagatagcctaaacatatgtttaatgtaattattatgtaagaaaataatgctgacgaagttacaggagctctaaaagagaaaattttattattttcggatactcgctttataataagtaattagtgttgccattagttgctatttttcatttttatatgtatgacaagacaaggaagatgcctattacttatattaaatattacagctattatttttatatctactagcgagattccgaaacaaacgaactttctagtcatcagctacataataatgatattattatcttacattttagttttgttatatacctaattgtattgcaagacttgaaaatataaacctacgtggctgcaggaaattttaacgaattttgaaataaactgccactgaaccgcgcgcctgttgaaagccatgcaagttcttgcacctacaaaatgtattatcttgtattttgactagacatagtctaagagattgacctggctacttcagcctatttaatttatattacctattcgaagtttggggcaaacttatccaattaaactttattaagtaattattttgtgtagacaccatacttccattcctatggaagatccacattaatatttatattgacttaataaaaataatctgataatacttacgatgtaagtaggcacacatttcatacttaagtccaagaagtattgaaacaacttgtcttttatttttggacgcctttctgtctgtgatatattatggaagcaggaagcagagaattggatgagcgatgagccatacatatactaacgtagtttttaattaataaatacataagtaggtaattattataattgaagtacaaagtttaagaaaatgaccatttaggtgcttaggtacctaactacttattcaacattcctgtacctaagtgcctaatacttaaataatatacctacaataatgtctgtgttgttttttatacctttttgttaaataaatacataagtttcatgaatgagaatttgtaccgaccaactataaactacataaaataagttatgtacatttatataatgtgattaaattttataaaagtttgtataataaaattata

SEQ ID NO: 4 - DNA, 1599 bp, Spodoptera frugiperda
  source 1..1599: mol_type = other DNA
atgtctgcccgcgcccacgccggcgccgcgctgctgctgctgctggctctggcgcgaccctcgcacgagcgagacgtgtatcacaagttatcacgcagcgtcgagaccgacaggctgaacattccggatgtagaacttgatcagcagttctggtacgatgaggcgcaggctgcaatcaatgcccggctggcgcacaaggagagcgtgaagaaggcgcgcaacgtggtcatgttcctgggcgacggcatgtccgtgcccacgctcgccgccgcgcggacgctgctcggccagcgccgcgggcacaccggcgaggaggcacaactcgcgtttgagaaattccccacggtcggactagctaagacgtactgcgtggacgcgcaggtcgcggactcggcgtgctcggccacggcgtacctgggcggcgtgaaggccaacattgccaccatcggcgtgagcgcacgcgtgccgcacggcgcatgcgcggcggcgcgcgaccccgcccaccacgtgcactccatagcggagtgggcgctcagcgacggccgggacgccggtatcgtgacgacgacgcgcgtgacgcacgcgtcccccgccggcacgtacgcgcacacggcgcaccgcgactgggaggcggacgccgacgtggcggccagctgcgcggctgcgcatgcgcgccaggacgacatcgcgctgcaactcgtgcacgcctaccccggggaccacttcaaggtgccacttactctaggaatcgctgctttaacagactgtggaggccgtcgagagtttttgccaaattctacaatcgacgaagaaggctcgaggggtaagaggttagatggcagaaatttaatcgaagaatggcaagagagcaagcgagcacggaacgccagccaccggtacgtgtggaaccgagagcagctgctggccgcggcggaggagcagcccgagtacctgctgggcctgttcgagggcagccacatgcagtaccacatgcaggccgaccccgccaccgagcccacgctggcagagctcaccgaggtggcgatccgttcgctgagcagaaatcaaaacgggtttttccttttcgtggaggggggacgtatcgaccacgcgcaccacgagaacttggtgcagctgtcgctggacgaggccatcgagttgagcgcggccgtggagcgcgccgcgcggctgctgggcgaggaggactcgctgctggtggtgacggcggaccacgcgcacgtcatgtccgtcagcgggtactcgcaccgcggcagcgacatcatcgggccctccgacgaccgcggggacgacggcgtgccgtacatgacgctgtcctacgccaacgggcccggctaccggccgccctcggccgcggggctgcgcgacgacatcactcagtacaattaccatgatgtgaactttgcgttcccggcaagcgtaccactggactcggagacgcacggcggggacgacgtggcggtgttcgcgcgcgggccgcaccactccatgttcacggggctgtacgagcagagccagctgccgcacctcatggcgtacgccgcctgcatcggccccggcagacacgcctgccgacactga

SEQ ID NO: 5 - DNA, 1632 bp, Spodoptera frugiperda
  source 1..1632: mol_type = other DNA
atgttcctcctgtcgctagcactggtcgcgctgacgggccccgcggccgccggcgtgccccctgccgaccgctaccaccccgctagccgacactctcccgccgggcctccggctcgagctcaggagaccgaggctagctactggtacgctgaggcgcaggctgcaatcaatgcccggctggcgcacaaggaaagcgtgaagaaggcgcgcaacgtggtcatgttcctgggcgacggcatgtccgtgcccacgctcgccgccgcgcggacgctgctcggccagcgccgcgggcacaccggcgaggaggcacaactcgcgtttgagaagttccccacttcaggtttagtcaaaacgtactgcgtgaactctcaagtggcagactcctcgtgctcagcgaccgcgtacctgtgcggcgtgaaggcgaaccagggcacgctcgggctgaccgccgccgtgccgcgctgggactgcgaggcctccaccgactcctcgcagcacgtgcagtccatcgccgagtgggcgctcgcagacggccgcgatgccgggatcgtgacgacgacgcgcatcacgcacgcgtccccggccggcgtgtacgccaaggtggccaaccggcactgggagaacgacgccaaggtcagggaggacggcgccgaccccgcgcgctgccccgacatcgcgcaccagctcgtgcacatgagccccggcaaccgattcaaagtcatattaggaggtgggcggcgtgaattcatcccaactacgtcacagggcaccagaactgatggccgcaacttgattgaggaatggcagaacgacaaacgttcaaaaagccttgaccaccggtatataaatactcgagcacaactcactgagttattgtcttcgccgccagactatctgttgggattgtttgaagacagtcacctcaactatcatatgcagtctaatcttacgacagacccaacgcttgcggagctaactgagatagcaatccggtcccttagtagaaacgagaagggcttcttcttgtttgtggaaggcggtcgcatcgaccacgcgcaccacgacaacttcgcagagctagcgctggacgagacgctggagatggacaaggccgtcgcgcgcgccgccgagctgctctccgaggacgactcgctcattgtggtcacagcagaccacgcccacgtcatggcttacaatggatactcggcccgtggacatgacatcctcggcccttccagagacttggacctggacggagtgccttacatgacgctgtcgtacaccaatgggcccggcttccgtccacatgtgaacggtatacgccctgatgtcaccgctgagagttattatgaagagttagagtggcgagcacacgtagatgtgccactggactcggagacgcacggcggggacgacgtggcggtgttcgcgcgcgggccgcaccactccatgttcacggggctgtacgagcagagccagctgccgcacctcatggcgtacgccgcctgcatcggccccggcagacacgcctgcagcggcgccgcgcatgcgctggcttcgccgatacatatagtggtcatggcagcgtttttaactgttttgtctcaaattaatattatatttaagatctga

SEQ ID NO: 6 - DNA, 1224 bp, Spodoptera frugiperda
  source 1..1224: mol_type = other DNA
ccacttataaataaatgataaaaatattgcgaaacatggcacatacccctgtcgatgatatccacgtaaaaccatattttcaatctgcttgaatttatcaaaatcacaggcacagattttccacacgggccaggaggcacggtatggagggcggggggcgacagtcacggcttctcagcaggttgttcagcagactcagaggcgatcaccacaagtacaatactctggataaacggttctcctttacttctaccgccatgagcgagagtacaggctgcagcaccaccatggatgaggacctggagatggatccccaggtctgctccaagacttccactccacacgcctatggacctgacgatgaccgctttcctgctaatatcgatttgagatacagtggacaatacttgacaccagagcaactgccggagttttgtgaccaactccataatctggtggaagtgataagaaatatacagtcgtatgccaaagtcacgggagagtacccgagtgaactagaacgtcggttagaacaagaagcgcgcgaagtagccgccatagctgcggaagcgcgcaacgcccgtgacgtagttacgcgtgtgcgtgcacaacgacgcgctgtgcgtgcgcacaagaagcatctcgtgtcgcaactggctagtttgagggaaaccgaggttcgggagttagagagcacggtgcgactgtcagatagaaagctcttcaagaattgggaggcgctgaaagactccacagacccagcggctttcgagcctttacttgatgaaatcaagaacaaacaagctgcgctggagtgcctgcttcgcctggtggacagcaagaaggccgcactgtctcgcaccgcaccgcctcccacaccgcttccaccacccgagtcatcactcgacactgatgtcgggtgcgactggcgggtcgtgcggtccgaccggccgcagaagacacgagggtcggcgctcggcgggcaagcggcggcgccgctcgccgcgttccgtgccgctctccggggagcccagccgacacgcaccgccgccacaggtcaccctgtacatccagggtacggagagcacctccaggtacagggtaggcagtgccggagcagtcgcccttacaccgctggagctgccctctggtggaagcgtcagggatatactcttctttaccactaactgaatacttgctttaatataaactacctgtataaaa

SEQ ID NO: 7 - DNA, 2074 bp, Spodoptera frugiperda
  source 1..2074: mol_type = other DNA
cattcattaacgaagtcatcagccgtgactaccgattctttacattaaatcaaaattcttgctcttatcaaatcttgatacgatcacaactgtctaagatttttccgtccttatcggacatccagaatttggacacgatagaaagggacggtgaatcgaagttttaggtgtggttaagaaaaattatgccgtcgtgcgtgccgcgatcagtgttcgtcggttcacctgacgtgcgggcgtgcgatcgatagcgcacggtgcggcactagcccgtttaaaattcgaacggtccgaattccgggaggccaatagccgtcgcgcgaactctttgatcgtagcgcaatagtgatgtgtgtgatgtaaaatcttgttgtcgatagacacagacacggtgatcgtgacaaacatgatgactatggatgtcgggatgtacaacaaccagcagaacgggtacgggagcgccgagtactaccagcagggtgcagcctatcagccgccatacgagggctaccatgaggggtactacgagactccaggacactattacgagcctttactacacggacaaacacacgagcatcctcctacacctgttataagtacagacactggtttatgttatactaatctagattatggtgagctgcaaccgaactatccgattcatacgttgcctcctcatccggagccgtttaagcacagagaagacgtgcccagacatgaggagatggtaatgcacgaacataagctagacaatcactatttggagacgaagtacaacatgcactttgtggatgagacgtcgatgcagtacaaccacgcggggtctcccctcccgtgcccggagttcgagcactaccagcctaaggatgagttcggagggctgcgggatgggttcccgcgggaggagtgcatgagctcgcacagccacctgcagggccaccaggcgcacgcgtcgcataccaccgtacccacgtataaatggatgcaggtgaagaggaacgtgcccaaaccagccgctcccaaattgatgattcctccgacggagttcgtcagtcaaggtggcatcggcagtccacaggaaggtctgcgaacgcccactggcagccaaatgctggccaaccctctcctcaacctgaacaacaccggccgaaccaacttcaccaacaaacagctcacagaacttgagaaagagttccacttcaacaagtatctgacgagggcgaggaggatagagatagcctccgccttacagctcaacgagacacaggtcaagatatggttccaaaatcgacgaatgaagcaaaagaagcggatcaaagaagggctgatagttgcccccgaagtcgcccccacctcgacctcccaacccagtgtcgtaggctccagtgagaacagccgcgaatctagctaaatccattgttttcaaagacgattattcgagtgaactgttggtcttattgatacgcaaaatcataacgtttttatcattttcaaaatgttgactagtacagaatagattatgctcttttgtgtggtttggtgtcttgatttttattgtgttcattttatttacgaatccatgtttagtagctagatcatattattacaatagttgtaatattactctgggaaaacatcacatctgtgtcaactctctgaaagcttttacaaaataaaaaatacgtgttgatacataagtttcgtaactaacgttcaaagttcgaatgagagtgcaaacaaacacgatgacaatttatacatatatctgcttatcctgagacaggataacaccagatcaaaaaaataataactgctgcaaataaaatataatttcattacccaatgaaaattagatagagcgtagattgtttttaatgaaacatttgtctttgaaaataaaacttatatcagaagagattattataatgttattgtaaaagatataaaggctcaattaacctgactagtgtaaaccaacggatgtgtcaacgtaatgaattttcttagaatttaaatgtaaatttcaaatcataagctaggtgttacttgtaagttcagccgctgttctact

SEQ ID NO: 8 - DNA, 3601 bp, Spodoptera frugiperda
  source 1..3601: mol_type = other DNA
tttttaattttcattctttttgcatttattttaatactttagaaagagtgaatttaaaataaatatatttttataaacaaacagtgattattagaagttagtatcctgattgtgattaaatttatggattgtttggcaacttcatcaggaaacggttttgccgcgcaaatattatcgattcgaggaagaatcagatgaggagacaccgtccgtcaacaccgagcagtccatcagtgagaccgatagctgttgtgtagtgaatggaatccgaagtgtttgtgtagtgactaaactgtgatagatagtgtcggcccgaggggccgagaatctgcaacggaagatacggacgcgagccgcgcgggctgcgggcccgcgatgagcgccaataactgcgacagtatgacctacttctccaacgcatacatgccagacatgaggaacggtggcggccacgaacaccaacaagcacacgcgcactatggcgctgtgccacaacaaggccatgaaatggagggctgtgaccagcagctcaggccggcgcagcaccactaccccgcgcagccagcacctggcatgccttaccctaggtttccaccctacgatagactgggttactaccaacagatggaacaaaatggctatcggcccgacagtccatctcaaatggggcacatgggtccaaagacagcagatggatatgggcccaatggtcaccagcctccagcgccagcggtgtatacgtcgtgtaagctacaagcagcggcagcaacagcgggaggcgttccaggcagcccgccactagaacaagcacagcagatgcctcatcatatgcatccacagcagcatatggtgcagcatggtgtgccgcatcagcagcaccttatgtaccccgtggacgacatgcagcaccagacgcagatgcctcccatgcatcagcagtccatgcacgcacaacaaccacctccacaacagcccccgccaaacactaacgcctcgctgcccagccccctatacccctggatgcgaagtcaatttgaaaggaagcggggtcggcaaacttacacccggtaccagacgctcgagctggagaaggagttccacttcaaccggtacctgacgcggaggagaaggatcgagatcgcgcacgcgctctgtctcaccgagcgccagatcaagatctggttccagaaccgccgcatgaagtggaagaaggagaacaagaccaaaggcgagcccggctccggtgacgagccggacaacatgagtccgcccacctcgccacaataaaggcctctagcttagttcgcgcggtccaagctaggggtgacttgtttcagatttcttcgcccaaatcacaaactccgttctcagcgttttattttgtgtatacgaattatgttcgatgcttcgaacacagatctgaaacaatacgccccgagaccgaagttactagaacagtttaatttgtataaattgtatgttggacttttcatgataatattacattataactaatacgtaacctagtgatttcgagtaatgatagtgtgatgaagagatttttacagttgttaagtataaattaatttatagaaacaatctcaataccaataattaccaaaaacgcagtgtatatgattagtaaactgcatcatttatgtgattatatagttgaataattatcattttctcggtattaactattgatttgcgatcggccacaatgtttgtataagtttttgacaaagtgcttgatactacatgtttcacaatatttattacatagcgactctaagttggcaacaatagcattttggtaagacaaaagaaatacggaaaacgtaacgtccataatttattgttgtatacgtatcgagcattgtgtcagaagttatgttaaactatcacaaaattctataataattttgaatcacaaaaccataaggacctcttataattaaccattgataagaaggcaattagtatccatattgttattatttaatatatttgatgttttatttcttaattaagctatctaaatactattctgtacatacaaacataataatttttagattatatgatgtacaattgttttagtgtgaataattaattattgagattaatttgggaaacaacgatattattcgtggttttgttttttgcggtatgagtgtttttttaatttgtggatcgtctctttttttctttactcgtaactcgtagtgcgggaaggccagtttacaagtgcaatgtatacgattttgatgacacatatacgtaattaatactgataataaacgtcataggaaatctattattattgtaaaatgttacgagatcgaagcttataatttagaaaatactaaatgagagcattaaagagatattctttgaaattctattgtgggaagttttgtgtcacgtgggctcatcttggacacgttgggagaaaatgatatgaatgggaaaggtcacttatttataatgaaacaaaagttaccttcaatattgtaggtgcagaacttgatactttgatttataaatctcttgtggaatgtaggttcgaggttacagttcacagtataccaaagttagaaaataaactggtgtttatacgaacgaatatacgtagacacggaggcgcttgacgtttcaagtataacagtaatacatcattttgaaaaggtatttttataaaatcattgattaagaaacccgatatacgtaaaaaaatatctaatatatatactcgtaatacgttagttttacgaatattattattataaagaaaaattggcgagtgatagaaaggttgatcttgtcaggattcaagttatacgttttttcgtaataatgtataaaaagtactatttgtaaattatttttcacatagattgtaattagtgaatgtttttaataagaactcagtgagcaggttctataaatattttttttcttttttttttaattttgtgctgtgttcgttaagtaatgtaaattgtaataattgtatattgtcgatatattcaatatttatttattgattcgctaattaggttcgcttacgttttcttcaattgcaattgggtggtatattcgacaactgtctggctgggatgcattttgttgaattatttttaattgctcaattcacatgtttgtgttttaattttattgatagaattaagtgcagtattttaatttgttgtaactttttaaaagtacattaagtatggtatatgaacgacattgtccgtttgagatcctccctagcattaatatgtatcagtgcaatgttacacaaaaattttcaatcccttgccagatggaaacagtgagtttgaaatgtaaaattttaaatttagttttacttttaaaattaagttctgattcttaagtctgtccctgaaaacgcaaaaattaggtctttagaatgacattggaacctaatttcaacgattttatttaccatttatgtcctatcgattcaaaatttatgaagtataactataattagtaacataataaggttaggcgagacgatattgtcataaatatataaatacatgaaattaataaagcataatgattgtac

SEQ ID NO: 9 - DNA, 2488 bp, Spodoptera frugiperda
  source 1..2488: mol_type = other DNA
tttttatgtgaaaagtgtcatcagataaacttgtgggatgtttagtgattttatcaacattgtgtttggtcgaaaagttggctgttgccgctgttggtcactctaaactttgaataggagtccatcaatcaagctccactctacggggacaattgaaacggaccgcgaacatcagtagcacttaacatgtccaactagactattctctgctcgtccaaacgatgacaagtgccgagtgatcttgagaagaaggacaaaatacgctgttatcgatgaacgaattaaattataacgcagggatgagctcctaccaatttgtcaactcgcttgcttcctgttatgggaatcaagcgcccgggcgtactgggacgcctgtggatcaagcaggccatcccggtttaccaacacctggagctgattactacaatccaaatgcggctgcatctgcctatccaaatgcttgttattcgccgccgcaagttggacatcactacccccagcacccatatggaacacctgcgactggagcgcacatgcaaccacaaacaatgatagactacacacagttacatccccaaagactgggcagttcggcaagtcacatgcatcaacactcgaatccgagtccaggagcattatctccgaatttgatgtccacaccaccaagtcaggctgctggggctagctgtaaattcgctgattctacatctactacaggagtagcctctcctcaggatctatctacttcctctggaccaggtcgaacgtcacctggttttggaggtgttggacagaactccagtggaaccaccagtaccaaactaggcctaactactcccatcgcttctccagtagaacacaaggctaatgtgaatcagaatatatcaagtccggcttcgagtacgtcaagcaatgagagcaatgaggctaataattcaagctcaaacacgaagaattcaaagtcttcagcatcaaactcctctgcaaaccctccgcaaatatacccatggatgaaacgagtacacctaggacaaagtacggtgaacgccaacggagaaacgaagcgtcagagaacatcgtacacccggtaccagacgcttgagctggagaaggagttccacttcaaccggtacctgacgcggaggagaaggatcgagatcgcgcacgcgctctgtctcaccgagcgccagatcaagatctggttccagaaccgccgcatgaagtggaagaaggagcacaagatggcatcgatgaacatcgtgccgtaccatatgaatccgtacggccacccctaccagttcgaccttcacccgagccagttcgcgcatctctccgcataggatcagtggaatttttcgaacagcggataactgatttattaatgtagacagtcgtccgcatcgcggggttgcgtgtaatatcttggttaacaacgatgttacagcaaccagactgggtcagttattcgctgttcgaaaaatttctttaagtgtagtctaattttgttattgttattgtgacgttacaagtgacaaatacagtgattgtgcggcaagataccgtgggacaggagtgacgagcatagagagtggtgattgtgaaaaacatgagtatagaacataatttaaagtttattccagtttcccatacctgtactaacgcatggaaggaaattatcgagaattccttatcacaaaaacgttaaatgtaaacattaactacaacggtgttaaaatttaaagtagagttgaattttacctagtcataagctgtatcgatttgcattattcgtaaaactgtaagatatgaacaactgaataccgatttatgtacattgtggtgactagagagtgtctaaaattaaatagtgcgacttgaaatttctggtatcgtttgcgtttcattgtttttatttcaaacagttgtcgtgtaggaaaataactcaatgaagtgctaaacgctgcccggagttttgtttgtcgtgtttctatcgtagacactttgatagaattaagatagactttaaccggcagatgtagtcatcacaagaataaacaaattcggaattgtacagggatagtgtaacgatgtacgaactacattgttaaagacaaaatgtatcagaaaatactataattttaggtcatttcattattagtaacgaatattagaagggggcttgtaaaagcgctgtgaaacttaaagaagtaagctcagttatagattgtgaataggtaatttcttcctattaatgtaaataacttgtcatgtagaaggtacggtgaaaaattaatatatatcaagtatgtatctcgaactgttttatgttcctgtctttagtttgtaaataacaaagtatttatcacaacataaatacatactatagttaaagttattaagtaaaataaattagtgattttttaaagtttccacaaacgataccatttaaaaatttcatgtggctctattgcgtactttaaagtcttta

SEQ ID NO: 10 - DNA, 2526 bp, Spodoptera frugiperda
  source 1..2526: mol_type = other DNA
atgggggccatgttccgcagcgaagagatggctctgtgccagctcttcatccagccagaggcggcgtacacgtccgtctccgagttgggagaagccggcagtgtgcagttcagagatcttaacccagacgtgaacgcgttccagcgaaaattcgtaaatgaagtgcgtcgctgcgacgaaatggaacgcaagcttcggtacatcgaggctgaagtacataaggatggggtgcacatacctgccgtcaaagaagcccccagagcccccaacccgagggaaatcatcgatcttgaggcccacttagagaagacagagaacgaaattctggagctgtctcacaacgcggtcaacttgaagcagaactacttggagctgacagaactgcgccatgtgctggagaagaccgaggccttcttcatcgcgcaggaggagatcggcatggactccatgaccaagtcgctgatatctgacgagacagggcaacaggcggctacacgtggccgcctgggcttcgtggcaggagtcgtgaaccgggagagagtgcccgccttcgaacgtatgctgtggcgtatctccaggggcaacgtcttcctcaggcgtgccgaacttgacaagccattggaggatcccgccacggggaacgagatatacaagacggtgttcgtggccttcttccaaggagagcagctgaagtctcgcatcaagaaggtgtgctccgggttccacgcgtcgctgtacccctgcccgccctccaacactgagcgacaggacatggtcaagggagtccgcactcggcttgaagatcttaacatggtgctgaaccagaccagtgaccacagacagagagtactggccagcgtggccaaggaactagccagctggacaataatggttcgcaagatgaaggccatctaccataccctgaacttgttcaacatggacgtcaccaagaagtgcctcattggagaatgttgggtacccacccaggacctgcccaatgtacagaaggctttggctgatggttctaatgcttgcggcagttcgatcccatcattcctgaactgcatcgagacggacgaggagcctccgaccttcaaccgcaccaaccgcttcactcgcggcttccagaacctcatcgatgcttacggagtcgcctcctacagggagtgcaaccctgcgttgtacacgaccataaccttcccgttcctgttcgcggtgatgttcggagacttgggccacggctgcatcatggccctgttcggtgcttggatggtctgcaacgaagttaagctcgccgccaaaaagtccaacaacgaaatctggaacattttcttcgccggtcgttacatcattcttctcatgggttgcttctcaatgtacactgggttggtgtataacgatatcttctccaagtctatgaacatcttcggatcgtcgtggagcgttccctatgacaatgatactatggaacacaacgcagccttgactatggacccgaaaacatcttacaacaacaatccgtacttcatcggtattgatcctgtttggcagagcgctgacaacaagatcatcttcctgaactcgtacaaaatgaagctgtctatcatcttcggagtgttgcacatgatcttcggagtctgcatgagtgtcgtcaactacaacttcttccgccgccgctattccatcgtgttggagttcctgccgcaagtgatcttcctctgtctgctcttcttgtacatggtcttcatgatgttctacaaatgggtcgcctacagcgccttctccgaagaacaagcctacaccccgggttgcgcgccatcagtcctgattctgttcatcaacatgatgttgttctcgagcacggcgccgcccgagggctgcaacgagtacatgttcgaggcgcaggcgtccatccagcgggtgttcgtcctcgtcgccctgtgctgcatacccgtcatgttgctcggcaagccactgtatctgctcgccgctggcaagaagaagaaggaagccaagcctgaacactcaaacggcagcgtgaaccctggcattgagatgcaggagcagactgacatcggtgacggcgcgcccaaacccgaggccaagccggcggcgtcaggccacgaccacgaggatgagccattcagcgagatcatgatccaccaggccatccacaccatcgaatacgtcctcagtaccatctcccacactgcctcctaccttcgattgtgggctctctcgcttgctcacgcagaactgtcggaggtgctatggaacatggtgctgacgttcggtctgaaggaccacgactacatcggcggcatcaagctgtacgtggccttctgcttctgggcgctgttcacgctcgccatcctcgtcatgatggagggactctccgccttccttcacacactgcgtttgcactgggtggagttcatgagcaagttctactcgggtctgggttacatcttccagccattctgcttcaagactattttagaacaagaagacaacaaggacgactaa

SEQ ID NO: 11 - DNA, 2705 bp, Spodoptera frugiperda
  source 1..2705: mol_type = other DNA
caaacaagtccgtgttgcgcacgatgagtaatgttcacttgaaagttattcatatcgattgatttgtctccactagtaatttggtttcgtcgctgccgaagtctgctcgcagactattgcgacggggcctttttctgaagttggattaatttttattttgtgttgagtgctgcaagaaatcgtgaaagatggggtcgttgtttaggagtgaagaaatgacattatgtcaacttttcctgcagagcgaagccgcgtatgcatgcgtgtctgaactcggagagctgggtctcgtgcagttccgagatttaaacccagacgtgaacgctttccaacgcaagttcgtcaatgaagtacgtcgttgtgatgagatggagcgaaagctccgatacttggagaaggagattcgccgtgatgggatccccatgcttgagatcccaggcgaatgccctgaggccccacaaccgagggagatgatcgatcttgaggcaaccttcgaaaagttggagaatgaactccgtgaggtcaaccagaacgctgaagctctaaagagaaactaccttgagctcacagaactcaagcacattcttagaaaaacacaagttttctttgacgagatggcggacccgtcgcgcgaggaggaacaagtgaccctactaggggaggaggggctcatggcgggcgggcaagctctcaagctgggtttcgtggctggagtgatcctccgtgagcgcattcctgcttttgagcgcatgttatggcgcgcctgcagaggcaatgtgttcctccgccaggcagagatcgacacaccattggaagacccctcatcttcggaccaggtgtacaagtcggtgttcatcatcttcttccaaggagaccagctgaagacccgtgtgaagaagatctgcgaaggtttccgtgcgaccctgtacccttgccctgaggctcccgctgacagacgggagatggccatgggagttatgaccaggatcgaggatttgaacacggtactaggtcagacccaagaccaccgtcaccgggtactggtggcagctgcgaagaacatcaagaactggttcgtgaaggttcgcaagatcaaggctatctaccacacgctgaacctgttcaacttggatgtcacccagaaatgtctcatcgccgagtgctgggtgcccgcacttgatctggaaaccattcagctggctctcaggaggggaacggagcgcagtggtagctccgtgcctccgatcctgaaccgcatggaaacactggaggacccgccgacatacaaccgcaccaacaagttcacgtcggccttccagaacctcatctacgcgtacggtgtcgccacctaccgggaggtcaatcctgccccgtacaccatcatcacgttcccattcctgttcgccgtgatgttcggagacctcggccacggcgcgctgatggccatcttcgggttctggatgtgttacaaggagaaaccactgcaagccaagaaaattgacagcgagatctggaacatattcttcggtggtcgctacatcatattgctgatgggtctgttctcgatgtacaccggtctcatctacaacgacatcttctccaaaagtctgaatatcttcggctcctcttggaggaacaactacaaccagtccaccctggaaagcaacaagctactgcaactcaaccctgattcagaagattatatccaaactccttatccgttcggtattgatcctgtttggcagctggctgaagcgaacaagattatcttcatgaacgcgtacaagatgaagatctccattatcatcggagtgttccacatgttgttcggtgtctgcatgtcgctgtggaaccatctgtacttcaagcgtcgtatcagcgtgtacgtggagttcatcccccagatcctgttcctgacgctgctgttcttctacatggtgctgctcatgttcatcaagtggaccacgtacggttccaccccaccattctacggcagtgatgaccctgagatcgtgaagacgagtgcgtactgcgcgccgtcgatcctgatcacgttcatcaacatgatgttgttcaagcacgacgccaacacgcgccccgtctgcgacgacaccatgtacgccggccagatgggcatacagaagttcttcgtgatcctggctctgatgtgtgtgccggtgatgttgttcggcaagccttacttcatcatgaaggagcagaagcagagggctcgccaaggccaccagccagtagagggcgcggccgagaacggcaacgcgggcggagccgccgcgcccgctgcgcatcatcacgacgaggacattactgaagtcttcattcaccagggtatccacacgatcgagtacgtgctgggcagcgtgtcccacacggcgtcgtacctgcggctgtgggcactgtcgctggcacacgcgcagctcgccgaggtcgcctggaacatgctgctccggaaaggtctgatgtcgacggacttccagggcggcatcttcctgtacatagtgttcgcgggctgggccgccatctccgtgtccatcctcgtcctcatggagggactctccgccttcctacacaccctgcgtctgcactgggtggagttccagagtaagttctaccagggtgagggttacctcttccagccgttctccttcgaggtgatcctcgactcggcgggacagacggaagaataa

SEQ ID NO: 12 - DNA, 1023 bp, Spodoptera frugiperda
  source 1..1023: mol_type = other DNA
ttgacttcaaattgattttctcattactggcaatatgaaggtcctggctgtgactctgttggctctggtagcggtctcctctgcgaggcacatcgacttcgaagatgtgatcgatctagaggaaatcactgcttacggttaccacaccaaggttggtggtccattggccgagaagatccgcatcgctgaagaggaagctgctcgcgatccatccaggattgttggtggttccaccgctagtctcggccaattcccataccaggctggtcttattgctgctatgcccgcatggaatggtgtttgtggtggttcgttgctgaacaacaggagagtacttaccgctgctcactgctggtttgacggacaaaaccaggccaggagcttcaccgtagtacttggttcagttcaactctacagtggcggcactaggatgacaactagcagcgttgtcatgcacggaagctggatgcccagccttgttcgtaacgacattgctatgatcaccttgccttctgcagtatctactaacaacaacctgaacttcatcgccctgccctctggaaacgagatcaacaaccagttcgccggcgcgaccgcaactgcatcaggcttcggtctcaccagagacggtggaagtgtgagcggagctctcagccacgtcaacttgccagtgatcaccaacgctgtatgccgcaacaccttcttcttagttgattctacgaacgtttgtaccagcggcgctggtggcagaagcacttgcagcggtgactccggtggtcctcttgttgtgaccagcggcggcagacgtatcttggtcggtgtcagttcgttcgtatctggtcgcggttgtcagtctggtgcccccggtgcctacgccagagtcacctccttcattagctggatcaaccaacgcctttaagcaaataattgtgacctgaacgacgccgtttaatggcaatgttttttctaagtcaaacagatttaatgttatgagatcaaataaaatattattatttattataa

SEQ ID NO: 13 - DNA, 1788 bp, Spodoptera frugiperda
  source 1..1788: mol_type = other DNA
atgaagtttttgggagtgattctattagctctagcaatttcaagcactgcaagacacatcactctagaagatggtattgattttgaagagaataccgcgtatggatatcatacaaaaattgggataccattagctgataaaatccgtaaatcagaagaaaataacctaaaaccatctagaataattggtggatccctcgtggatctagaagagtttccgtatcaggctggtcttatccttcgactgccaatcgggacgtcaatcgctggtgcagtcttaatatctccgaatacagttttaactgttgctcatatttttaatgatgctgtattatgtgtgacaacagtcaccgtggtactggatacaatctatatattcattgcaggaataagaattgatacagacgaatacattttgcatgaaaattataatcctgctacacttagaaatgatatagctcgtatatagtcgtcaaattacgttataatgttgcttttactgccactgtcagtcctgtgtatttacccttggaatctatagaggaagaagatttaaatggagagactgccatcgcttctggttatggacaaacctccagtagtggcatctcaataccaatgctgttgaattctgtaaatctaactatcatgacgaacgaagagtgcgctagagtttttggcaccttcatccaacctacaaatgtctgtgccagcggtaatggcaacgtgtgtcagggggacgccggtggacctcttgttgtctacagagacatgagacattttttgataggtgtgtcgtcatttggaacaggccgatgtgaaactctttctccatccgtcttcaccagaattacttcttacacagattggatcatggaacgtattatttgtaacaataattatgaaggtcttggctctagcatttttcgccctggcggccgtggccaccgctaaaaacatccatgttgaggatgtcatcgacttggaggacatcaccgcctacggttacttgtccaagatcggtaaacctctggctgatgaaatccgtaaagctgaggaggaagctagtgcatccagaattgttggcggttccttctccgagctcggacagttcccttaccaggctggtcttctcgctgacttctggatgggtcagggtgtgtgcggtggttctttgatcaccgctaaccgtgtgcttactgctgctcactgctggttcgatggtattgaccaagccttcagtgtcaccgtagttctcggctcaatccgtttgttcatcggtggtgaaagaatcaccaccaacgatgttgtcatgcactggggctgggaccctagcttgatccgtaatgacattgccgttattaggttgagctctaacgtcgccttgtcagatgtcatcgctcccattgctatcccaagcggtgacttgatcaatgaaagcttcgaaggtcaaatcgccgtcgcctctggattcggtcgcaccagtgacaatgttggagtctcaatcctccaggtgcttagtcacgtcgaactacccgtgatctccaacgcagtctgcaggctttccttccccctcatcctacaggactccaacatctgtactagcggtgctgatggcaagagcacatgccgtggtgactctggtggccctctggttgtcaatgtcgatgacagccctgttctgattggtgtcacttccttcggatccgcccgtggttgcgaagttggtgcccctgccgctttcgctagagtcacctcttacatctcctggatcaacaacctactctaa

SEQ ID NO: 14 - DNA, 789 bp, Spodoptera frugiperda
  source 1..789: mol_type = other DNA
atgaagttcttggctctagcactcttggctctggcggcggtggccaccgctaaaaacatctttgttgaggatgtcatcgacctggaggacatcaccgcctacggatacttggctaaagtcggcaaacctcttgccgataaaatccgtattgctgaagaggaagctagtgcatccagaattgttggtggtcaggcctccagcctcggacagttcccctaccaggctggtcttctcgctgacttcagcttgggtcagggtgtgtgcggtggttctttgatcaaggccaacagtgttctcactgctgctcactgctggttcgatggccagaaccaggcctggagattcaccgttgttcttggctccatccgtttgttctccggtggtaccagagtactgtcctccaacgttgtcatgcacggaagctggacacccaatctgatccgtaatgacgttgctgtcatcaagctatcctccaacgttgctctctcaggtgctggtatcactgtcaaccagttcctgagccacgtcaccctgcccgtgatcaccaacattgcatgcagagcttccttcccccttatcgtccaggactccaacatctgtaccagcggcgctggtggcaggagcacttgccagggtgactccggtggccctctggttgtaaccaggaacaacagccctctcttgatcggtgtcacctccttcggatctgcccgcggttgccaggtcggttcccccgctgccttcgccagggtcacctccttcatcagctggatcaacagccaattataa

SEQ ID NO: 15 - DNA, 2677 bp, Spodoptera frugiperda
  source 1..2677: mol_type = other DNA
cgctactgacttacctagtggccgccgtggtggtggcggcgtgtgtccgcggggaccggtaccaccccgcggaccccggcagcagagctgacaccgttgtgaaccgtgccgagacctcagccaactactgggcccaagaagcgcaggctgcaatcaatgcccggctggcgcacaaggagagcgtgaagaaggcgcgcaacgtggtcatgttcctgggcgacggcatgtccgtgcccacgctcgccgccgcgcggacgctgctcggccagcgccgcgggcacaccggcgaggaggataaactgcattttgaaacattccccaccgttggattgactaagacgtattgcgtgaacgctcagatcccagactccgcgtgcactgctactgcgtacttatgtggtgtcaaaacaacttacggagctattggagtgaatgcggaggtgccacggaaaggctgcgaggcgtccaccgacaccagccgacacgtggagtccatcgccgagtgggcgctggccgacggccgcgacgcaggtatcgtgacgacgacgcgcatcacgcacgcgtcgccggccggcgtgtacgccaaggtggcggaccgcaactgggagcacaaccaggcggtggagaacgatggcttcgacacggacaagtgcccggatatcgcactgcagctcgtgcataagcaccccgggaataaactcaaggttattttaggcggaggaagactaaactttttgccaaatgatgtgaaagacgaagaaggggtatatggaaaccgaacagacacccgcaacctcatcgaagaatgggcacaagacaaggaagatcgtaaagttactcataaatatgtttggaatcgtgagcagctgatgagtcttaaagatgatcttcctgagtaccttttaggacttttcgaaagtaatcatcttcagtacaacatgcaggcagatcctaatactgagcccacgttgactgagctaactgagatagcaatcaagtcgctaagtagaaacgagaaaggttttttcctgttcgtggaaggcggtcgtatcgaccacgcgcaccatcgcaactgggtagagctagcgctggacgagaccctggagatggacaaggccgtcgcgcgcgccgccgagctgctctccgaggacgactcgctcattgtggtcacagcagaccactcccacgtcatggcttacaatggatactcggcccgtggacatgacatcctcggcccttccagagacttggacctggacggagtgccttacatgacgctgtcgtacaccaacgggcccggcttccgttcgcatatgaacggtatacgccccgatgtcaccgctgaagacggtttcggagaagacgaatggttggctcacgtagatgttccgctgatagacgagacgcacggcggggacgacgtggcggtgttcgcgcgcgggccgcaccactccatgttcacggggctgtacgagcagagccagctgccgcacctcatggcgtacgccgcctgcatcggccccggcagacacgcctgcagcggcgccgcgcatgcgctggcccagcctgtgctgctgctctctctccttgtactgctcacttcactattccaacaatgatctttaacattagtgtatgctgtgattatttaatttaataaatagtaattatattataatacaagtaggcctacataatgggaccaggcttataaaaaattagactattgctagaatttaattatttggtatttagggaacagagagtaaagttccctaagaaaaggaggatcctccgaccaggcgaggtgttcagcctggggggctttctgcccaactgttgttcgttgggattttctatccatgttaattcgcatgtaaacttcatatgtgcgatgcaggatatttatgacgtcatccgttgattggctcgtagatagtctatgtgcgacttctgtcatctgtcacttgtcacttgtcagtgtcgccacaggtaaaataaaattcataaaagacaattaaataattacaattataatgaaactggcgttttctttaagttccctactcaaaggataaaaatgacggttttatatgaatgtaaaatttaagattacggagcttgcgagaagagaattctagttctgacagtttgattattggtgcgacccacccaaatgttctgttggtaggtattgttcgtcgcatcagtcagctgcatccgttaattgagctgatggtaaaccgatacatgcgtactgccatcagcacgggtccgctcaaactggtgtagctctttcctcaaaaggccatgaacatatccctaccttacactctcacgcctgtacaccatggacctgttcacctcaatcaagcaaaatattgacgagttgaagcggcatgacttgaagaaatttgaaccttatgactccaatgttatttattcaattatatttgtattggtgaccttgctacccatacttgcatgctgcaacctgagaccaaggagtattcatcccatgaatgggcggtcaatgaagacagcccacctactgccgctccacacgaccggagttcagtagacgagataactgtgacctctccaccgccttctacgtgtgacgtagtaattaggcattaggacttaagaatataacctgtatcttagaataggagag

SEQ ID NO: 16 - DNA, 5035 bp, Spodoptera frugiperda
  source 1..5035: mol_type = other DNA
atattttattatacttcatttccaagttcttggttgttaacggacggtgatcacaattagacggctatggacactggcatgacagggcattcaccgcatgctatccctgggcgctcctggggaggtcttaacgtagcgtatgaactgcaagatccaaacaaaccaggtatgagtggcatgcctggtcctagtaagcagcctgacatgggccccgtgaacatggctcccatgccgcctacgactgtacggaccaagaaacgcgccgctatggctacatcggtcgtcgccatggctaacacagcttctgctatacagactatgacgacgcaaacgttggccagtgtacgccgccagcctcagccgacgccaccgccgatttactcagagactattaaagactcgccaccgagctctccaagctctgaaagaccattaaagcctaagatggagtccaaaaccttcatgtatcccaggatcggagcttcttgtaccgccccacacatgctaggcaatgagcttaacccagagagcggagcggccgcttgcctccaggaacagttgactgctgaactagccgcccatgccgccggtagcgcaggtggggataccctcataccgccgccgcttatcaacaaagccgcgcccggcggcagtatcagctcaggacggtcctcgagcgctcaaagcctcgaccagttgctggaacggcaatgggaacaaggctcacagttccttatggaacaagcccaacatttcgacatcgcttcgctgttgtcatgcctgcaccagctgcgcactgagaacgttcggctggaggaacatgtaggcagcttgctgcaacgccgcgaccatctgctcgcggtaaacgctcggctcgcaataccgctcactactgaaatggtactaggagagagatgtacgaattcaagtgatgtgccattgcgtagtgacgactggcccgcccgagccgagccggtgcacgcgcgaaaatggcccgcagatgatgcaggtgcggccgccgccgccgccggtccgcgccgccgagactatcacctcggaccgcccacaccagcttataatgcgtgaggtgcagcagaagcccagctgaaaggaagagggtggcggcggaggcggtggcgatgctgccgtcccggggccgtcatcggccaacccggcagcgctgcacaccgttcaaacattcataatgctcgagcaacgacgcagggtcgatttctccatcgactccatcctcaagttaaacaactaaatcgtaggcgcgctcgcttttagaagccacaatgaatattacgttgttctaggacaaggcttctactgcaattataaagtgcgtctgcgttgttagctcgtgaaggagcaatgttgttgcgcggctccaactcaatgccaccacccgctataaggactcagctcgacggccgtcggcttacggacgatcttgtgttccagctcctcggggctgtagtatacactactagagttaaagttcttaatgttaaactagattaacgcacggtgtgtgttatatttttgggcagtttcagcgtttatacgccgtgtcgaggttgatatcttgaatgtgatggtctactacaaagtacgaaaccgatgcttatagtgttatataaattgtttgcacattgtctatcgtaaagtactttataagaataagtgttcaagtttcacatacgcttttgtatgatacggtttctcgagttgccaactttttttgcccgattgttaggaggcgtcctgtacggaccaaagttggtaacgttccgctttatctgtagaatacactgcgggatgtgcgcgccgcagctctggttggtactttggtttagatctcaggtcacggcaccaccgactgagtagattatgcgaagtgcggtttattctataagtgttattgtatattcgactggattaatatcttgatctatttgaactgtctgttcttttgaacgacgcatgcgattaaatgtttaattagaattaagtactaaaactgtgtatgtacctaggtatgaataatatatgacgaggtaggaagtcactttctactagttgaagattgaaattaatgttctgggaggattatattcctcccttgaagtccaaaagttaataaacttgtttgctggggatactgaacgttttgtgtatgatataaataaagtaatagattatgtagcactgaattgatcgagtatatcggagacgttggcctggacacacgttgggaatgatttcaaacgggaccgttacatatattgatgcatgtttaaattatttattataaatagttaaattgtgtgttagttgttaattgtatatgaataatcttgatgaatatttacagtgtttcgattgacgaccggcacacagcttggtaatgttctttgacgataatttcgcgcacagagcagggccgtgttcgtataagcggtatatgtttgttaccgcctttttaactatatctgaacacttgtagttgtaaggtcaaagcaaggatttaaatagttatagaagtaattctcattcgaggacccattcttagccgttccctgtctgttttagatttgtaacaatagttgtaggtgtctacagcgcgagacgcaacctatttgatgtctgcgccacatattgttgagaaacgcgacacggccctgatctaatataataactatttattccttaacatagtctacgagcacagatacagtatttttatataataaattcacgatgtgatagagtacgttcctcaaaattgtgtataggcgtttggctcagattaatatattgtggggtaaattattgctgcggtcttgataccgattttttgtggcgtgacgaccattttcggacagcatcgagtggttgttgtgtggactgttgacgctcgatataagtgtatgtgccggcaggacccggctaatgagagattttcttataattcggcactgaaccggtagtctcatcgccgtgatgtgtctttgtgccggtggcgttaaatacgctcaattacagcgttgttcgtcacgtatatacgtaacacccactattattatacggctgtttaatacgtcgttatattaccgttgtctcgcgtaacttaaacaaatcaatgtatcgattatattgtcataatatatgttgaatgttgttgatcgatgtacaactcttatatctcgatgttgaatgtaaagtggaataatatttgtttcaagatatcaatattaaatgtttgatcgactagtactcgcctactcaccaggccagtcactccctgtggtcactggggccgtcttatagctttcctcgctgaagtaagggcttcctgtgttgtccatgcaattttaatgcagttatagttaaatcagtataaattatattcattgttgataagtcgatgttgttagttcgcataaagaagtcgtgtgtcgcgggacggtattgatacagacctcggtcttgtaccacgtggcacacgagctctccatgcatcctcaaataatattaaatggttaaatgtcagtatgtaggatcttcaatcactggtgtagttcctttatctccccctttgggatagtacttcggaagaaacttcaatggaacaactcaattaaagtttcttcgagacggctctaatattcacttctaatttccctaatatagatacagcttaaacgtatttacatgtttcaagactaactgaaatctgataggtatctcccagtacactgaaaacgactcattttatttcaaaattaggcaattatacacaaatgtaatttttaaatttaattcgtaattatgtttcaaatcatgacaagttttaaagtttttaatattactgtgaagcgatgacttcgaagcagcttctttttactttaatattttaacttgattttacgcattattcaattgataaaatgaaaccttcatcttaattattggcgactattgtcactatcaaattaataaactataaatttcaagtattttgatagaaaatattctaatcaatcgattttaaacttaaaattttactaaatgttttcagataaacttatatatcaagaagtatatagaattttaactacattttaacaaaatatataacagttatgacattgaagatatatgccgatttgaaacataattttatcttgattaatgaacattgtttgaactgacggtgtgcaatattgttaagggataaagcagaatattgaaacaagggaatacaatatgcaaaagttctacaaaaaatattgaagcatttcctacttaatgaaatatttgtcatcatttttcttaacccttattctgcttgggaattgctaaagaaatgatacaagtttaaacaatgtttattagttgttccgatatctacaacattgtatatttaaggtacgcttcatttccaagcgaaggatacttttgttgtaaattatgttgttttaagtattttaagttttatacattaagtgtcaatgttttttaaccgaatcgcttggatgcggggctcagcacgttttgcatacaaatgtaaacagcgtagatttagtttcgttgggattggatcaaagagctgtgctgaatgttttagaaaagtgggaaatgtaggttacatggaataataccatttcattactctagtgcacgtttcgtttgttcagtgcagtttacaaagagtattttaatgcataccttttagcactaagctcagcctactgtccaatgtaaacgagactgggactacgagcctctccgtctaacgcgagggtttggtgaatattttcctaggatagtgcgcttagaccattgcaaacttcaaacgaagtgatctcagtcgatagtactgatatttcacgaaatcatattcctagtgtcgcttgtacaatagaaatatgatctcgttattaagggaatcgtaacgaaaattatggactgaattgtttataattagttttaattcgttaatttggttacatacatatgcaaattt

SEQ ID NO: 17 - DNA, 1697 bp, Spodoptera frugiperda
  source 1..1697: mol_type = other DNA
gcgcgcagtgtactcgccttttgtgctttcatcgtcaccgtacgatccacgttacgaacggatcgtcagttctggttggaccttgcacacaatgagttagaagaagccctgaaagtgaaatggaacctcaaccctgcgaagaacgtgatcctgttcatcggagacggcatgggacccaatactgtgacagcagccaggatatacaagggcggggagagccacaggctcgtgtttgagaagttccctcacatgggatttttgaagacatactcagcaaacaagatggtgcctgactcggcttgcacagcaaccgccatgttcagcggagtgaaggtcaaccaggatacagtaggagtggacgctactgttcagcacagggactgcgaagctagtctccaggccgaaaccagactgcagtctcttgcagctttagccttagacgctaataagagtgcaggtttcgtgacgacaatgcgcgtgacgcacgccacccccagccccctgtacgcacacagcgcaagccgcagctgggagtgcgaggccagcctgccaaaccactccccctgcaaagacattgccaggcagctagtcgaggactggcctggacgagacctgcaagtcattatgggaggaggcaggcagcagttggtatcgaatgcgacaggcaccgacgaggaccctataagcacgagcccctggacctgctaccggcaagatgggcggaacctcatcgagcagtacaagactgacaaggagacacgaggtcttaaacacagcgtggtcatgaacaatcaggagttaagagagttgaatgtgaatgagaccgactacttacttggaatattctcgaacgagcatttgagttacgagcacgaaagaaacaagggaccggagggaatgccctcactggctgagatggtgagagctgccatcaaagtcctgcagaagaatgagaacggatacttccttatggtagaaggtggcaacatagacatggcccatcaccggggctgggccaaggtagctgtgagtgagacattagccatggagaaagctgtgcaggtggcagccgacatgactgatgaggaagacaccctactcattgtcaccagcgaccacacccacacactgaacataaacggctacccaaatcgagggagcagcatctttggagacaaacgagcaaacgagtcacttgatggtaagcgatcagcgccgcccatggacacccgcaacaacacagtagtcaaaggcatagcccaaccctcgagctacgatggagtaaactacacaacgttatcctacgccacgggaggtccagggtcgttccagttctatgtgcagtcggtcccgactggcaacatggtgacgagacgagaccccagcacggaggacacggagcagtacaactacacacagatcgctggcattcagctcgtggagaattaccacggaggtggcgatgtcgctatctacgctagaggtccctactcccatttattccacaatgtccacgagcagcactacgtgttccacgcgatatctcacgcagccaagttcggcaacgagtccagaagctcagcaagtcaagtcagtgccttccatctgaccatcatagcagccatcagtttcttcatgctgtgacgacgcaaagcgaaatagattctaagtcttagtagataatgtctactttagaa

SEQ ID NO: 18 - DNA, 382 bp, Spodoptera frugiperda
  source 1..382: mol_type = other DNA
aaggagttacgaaagcaacctacgatgcctaaaattttgtctttattactttttgtatttattgttttcatttgccatgggcgaggacacagtttcgctgttaatcagttaataagcgatgcggatctccagtcagcattgatgccccaagaattgaatccagcctacgttcctgaaaaggatgcaccgtggcgcgctgtctttcccctcgggcgactgcgacgcgacatcgaagtcgtggaaaacgcacaggcacctgcgaccgtgtcgcatagtctgaaagtgtacagtcgcatggccacgtgcatcgcgctagcaatcgcagcgattgtcgagattattttgtatgtaatcgagttttatactgtcagttgaatatatt

SEQ ID NO: 19 - DNA, 4731 bp, Spodoptera frugiperda
  source 1..4731: mol_type = other DNA
ttggacatgttattttactgtcatgcagtgtgaaacataaataaagcatattagtttttgtaaaaatcatgacttaatactcaggcctaaactgttaatatgaaataataaatttaataaaataactgccacttacatatttgttttaataaatgacattatggatggtttaattcacgcggcattggaagctgaggtaatactaaaccgggcgaaacatgtgaactcaaacttatcccaatataacagtggtgtcccagaccacaaaatgacttggacccatgataagatgcaccttgctgaatctgttgatgaatctagaatgaaatttcaacgaaattcgtccggctctgcttccaaagctacagagaagtttgatttatttaaaaaacttcatgaactttacaacgagttaagtagagatgaaacattacaggtgaactatcaaggcctgaaaaccacatcatatctattggagaagttattagcaatatataatcttaatacattaataataaatctgtatccaggcaataagggatattctttatcattaaaagtaaatggaaactcacaacatctacatccaccagatgggcattcatcatcaaatcaagaagagactttgatagagactccccgctggccatatgaagaggaagagttattgagttatattgacaatgaggaactgcctgtagtgttgttagatcttttggagactgaacactcatgcctgttttattccggatgcataatagcacagattagagattatagacaggcatatccaagttttctctgcgatactcatcatgtactcctaaggcccactaatcagagtataataaccgatgcaatgtgtatcggtggtcgttgcggctgggcgggtgaagagcgtggagcgttagaagcggtagaggcggcactagtgcacgcggccgcgccccccttgtgcctcgacccgcgccccgccgtgggccttctagccgcgcgtctacatgctgccccacgactgttcaatacgccccggataagaaggcaggctaaacgattctcacaggtggcagtaaaccgaaagaggaaattggatcagttcacacattatcatggtttagagatattagacttaatacatcgtcaaagagcgaaaaatagtagacaatcagtgccactaacacgactgacatctaagtatccgaaaaagccaccagaggtgttcaaaccaatcgagccgccaaaaatggatccgttatcactagccgtgccgtccgagccaggcggtccgctgcgactggcccgcgcgtacgagcggccgcggccgacgcccgactgctacccgcagctagtggaggagtacatccttgagactgagaagacgtcaccacatgccagcgctgggttcttccacatcaaactgtcaatattacagagggccgccgaccaggagttccttggagaactctatgtagacagggaccacgttgaaggagaaacgaatggcgctgcatgtcggttttcattagggtcaagactacaagcgaataaatatatacagcagttcacagagatatttacggaggagggtcggaagtctgttaggataagacatgtagtgcctggccagatgccgagggtctcttttacaggaggcatgagggaactgaaccagcaactcttgctacaacagaggtcggccgcttctggcgccacgacagtacagactaacgccacaaccgttcctgttgtgacttctacgatacaggcaacaacgaatacccataccaacgtccgccaactacccatcctgcaagcgcagttacagcaagtgggtaatgtagcagcggtagggaacgtggtgagcaatgtgggcaacgtcgtgggcaacgtgagcaacgtgggcaacgttgtgggcaatgtgggcacggtggtgggcaacgtagtgggcaatgtgaccgccaccaacgacacggcgctcaagcaacaaccctcccctacgacaccacgactttcgccacaggtgcgtatttgtggggggaacgtggtcggaagcgtgggcaacatagtgggcaacgtgggcactgtcgtgggcaacgtagtggggaacgtcatcactactagtgacaccgcgataaaggaacagcccacacttgcgacaccgctgctatcgccgcaggcgtccaccaatcagctgttagcgcagcagttgacgaacccaccgcagccaatcaacccgcagaagatgcagtccgccatcatacacatacaacacccgctcatgtccactgccggcacgtctcaggtacagaacatccaatacacgaacacgacgaccacgcaacagaaggcgaccataaccaagcccagaacaacgaacccagccatcagcgcgcttgtcactagtcttatgaactctgcgcaacagttccaacaagcggcagccagtcaaaacgcagcgaaagcagcagtcagtacagcgagcagcaacgcgacgatactgaatttattgaacagtgcgccggccgccatgacgcacgccaccgcatcgactgcagacaataacactatagatgcccacaaactactcggccgggtctctatagccggagcacgaatcatcactgccactaccgcatcacataccttacccacgtatacgcaacagggagttggtgtccaagtaatgagcggttactcgagagagaacgaagcgacgaatgtgtcgagtagtgaaagtgctctattagagcgactgatggggcccgacacttcggccgcgcccactacgcagcaacaacccgtatgccatttgcagggcttaagtttagcgtcacttcaaggtctacaaagtattcagggtctccagaacgtacaagtacagatcccgggtttgtctgcgccgatatcgctgtccttgaacgtgtccggtgcgcctagtggtctgctagtgtcggtgccccctactacgtccgtcgtactcacaaaccaaccttcggtgctctctttgcctatagcgcaattgatgtcaggtggcgtgaagggcggcgtccgtggcggcggcgtgcaagtagtgcgcagtgcgcggggtgcgcggccggccccgcgccccgcgcgccccgccgcgccctcccccgcgccccccgccatgcccgcgcccactctcacgccggcgccgcatactggaacgacacaattcatcagccaggcgcaagcgcaggcgttgagcgctcaacaagttcggcggaaatcgaatgcggacagttcgtagtgacgtcacagtgtgcgacgcgacgcgacttagtgaaatattgtgtaaatattaacccctagctaaattattacatatgttagtgacatagtgactagtgactagtgccgtgcgcagtatccacgctgtgatctacatacgtacccggcggccatctctaaacaccgatattcgagaaatagacttaacttagtaagtgcgtttatgcaatagtggaattatggttgttttaatgatacgtcacgcagttgttataaatccaattaggagtaactttggttaagttgcggtatgtacgtaccgttaatgttttctaatacataactaggttcaatataaaaatttatatataagtagtatcctcaatgttgctatatccaatataataatgtctgaaacaacgactgaatttttataatgtagcagtgttcattgaatcagttatgcctatagttaaattatcagttccatcacactacatgtaaatggtctattaagtatttgcaatgatttgtattgaatcatgtctttgacaaattttattttattcgaattatttatcatattatactattttcaatcatgtatttttattatttgaatgtcacaaattataatatttattgcgtatgaaatttaattattaattgcaacattgaaagtgattggatcatgacatgaattttattgtgaaaataacgtttaactgcatagtggaataaaataatattgttaatgaatccggatattattatcgtaatgaagattttatgtacgacacctgtatatgaatgttgttagtgagtttgttagattaacaaatttagaacagcacacaggttgaattatgtattcagtaaagtaaattgagctaacgtgattacgtgtggagacttgactaacatctacattgtagaaaattcgagtaaatcagtattaataagaataaaaatcactgttcgcatgacaacctagtactgacgtttggaactttatgaagtacaacactttgcagtgattcatgaatctgtgttaggtaagatgtaaatatgtattatatagtttcgagtaagcatcacagctaatggtagttggaatgtaagaaagttgaatttatgttttcagtatgttactgtaatataaaatatgtgaatagtaaggaaatttgtttatgttatgccattgggaccgagttctgtagatttcaaacctggactataaggaatagagttgtttttattttactaaaagagaattcatatatagttttataatgtaatcaaaagagcaatatatctgtttaaataactatgcataatattataatgatactgtattatctcgaaagtatttaaaattagcgctcttagtattagggagacagccttataaatctgacttagatctaaatacgtaatactattgttatagtatatatttgttacctctatgatagaacgtgt

SEQ ID NO: 20 - DNA, 1655 bp, Spodoptera frugiperda
  source 1..1655: mol_type = other DNA
aggatgtttcggctcatcctttgcctggcggccgtgactctggcactggcctacaaaaaccctcactatgcatcaggccgtacgactatggttcacctgtttgagtggaagtgggatgacattgcaagagagtgcgaaactttcttaggacccagaggatatggtggtattcagatctcccctcccaatgagaaccttgctatctggtctcgccaacgtccctggtgggaacgttaccagcccatctcctaccgccttgtcacccgctccggtaacgaacaacagttcgctaacatggtgcgcaaatgtaacgacgctggtgtcagaatctacgtcgatgccatcatcaaccacatgacaggcacatggaatgagaacactggtactggtggcagcaccgccgacttcggcaactggggctaccctggcgtgccatacggcaggaacgacttcaactggccccactgtgtcattcaaggccacgactacggctgctgcgctgatagggtacgtaactgcgagctgtctggtctgaaggacttgaaccagggcaatgaatacgtccgtcaacagatcgttaactacatgaaccacctcatcaacttgggtgttgctggattcagaattgacgctgccaaacacatgtggcccggagacttgcgtgtcatctacgacaggctacacaacctcaacactgcccacggtttcccctctggcgctcgcccttacatctaccaggaagtcattgaccttggtggtgaagtcatctcccgcgatgagtacactcctcttgctgccgtcacagaattcaaattcggtatggaactcagccgcgccttcaaccgtggaaaccaactcagatggttgcacaacttcggacctgcatggggtcttcttgcttctggcgattctcttaccttcattgacaaccacgacaaccaaagaggtcacggtgctggtggaaacatccttacctacaagaacgctaaacagtacaagggtgctattgccttcatgttggcccatccttacggctggcctcagctgatgagctccttcgacttccacgacactgaggctggacctccaatggacagcagcggcaacatcatctctccctccatcaactctgaccagtcctgcggtaacggatggatctgtgagcaccgttggcgccagatctacagcatggttgccttcagaaaccaagctggcaacagcgctctatccaactggtgggacaacggcggcaaccagattgccttctgcagaggaaacgctggtttcgtcgctttcaataatgaatactgggacttgaacgagaccctccagacttgcctgcccgctggtacttactgtgacgtcatctctggtgagaagagcggcagcaactgcactggcaagcgcgtcactgtcggcggcgacggtcgcgctcacatctctcttggagccaacgagttcgacatggtcttggccatccacactggaccagagtcgaggctgtaagagtgtaaagaagtattccagtaattaaacatcaacatgaaccatcgattacttattaaatctgcgtttgtttaagtctgtgatttattattttaattattaagaacctttattagtgatatatatctaactataaaaaataaactata

SEQ ID NO: 21 - DNA, 606 bp, Spodoptera frugiperda
  source 1..606: mol_type = other DNA
atgggccgaccccagcccatgtcggagtccgacatgctcggactgatgcggcgggagatggctgctttccggcagcgctttgacatgctggaaagtaaggtcctccgtccacctctagcagcgaatcagacggcctcgcgctcatatgcgactgttgcggccaagccttcgacctcttctagtcagactggccgctgcactcagccgccggtgagggcaagagcggctcctcctgcaagggccccggtgcctccatcagtgcaggtggaacctccaccgaatgcgccaacagggcgcagaaataaaagaagagggggagtaacggcgcagcaggccgttggcccggccacagttgtgcctatcccgccggcaatagatctcggtggtggcgagcggcaagtagtcggcgaggcgaggagggtcgccaaagaaggcaggaagcgccgcaaaaaagcgcagcggcagcggcgccagcagcagcgcaaagagaagcgcgctgcggccttgcttcgcgcccctaagaccgcggctgtagttctaacactacagcccgatgctgtccaaaggggcgtgacatatcgacgctggaaaggcaaaatgtattaa

SEQ ID NO: 22 - DNA, 1861 bp, Spodoptera frugiperda
  source 1..1861: mol_type = other DNA
ttatcacgtaaaagatcgaatctatatccaaaagaaatacatatctttctgaaactttacctaatcatcagtttaagtggtgtaaagtattgatatggcctaggattattacttttgtcttgtttatcggtaaataataattgctacactcctagctacatgtttattttcatgcgaagcttattagtactgtgtggtcattaattcgataacgttgcggaaagatgggcgggctgacattactctgcagcctggcgatcctcgcctcggccgtaactgcgtacaagaacccttactacgcgcccaataggtcggtgaacgtccacctcttcgaatggaagtgggacgacatcgctgatgagtgcgagaggttccttgggccaagaggttttggtggtatccagatctccccgcccaacgagaatgtggtgctatggggctacaaccgcccgtggtgggagcgctaccagccgatgtcgtacaaactggtcactcgctccggcaacgaggcgcagttcgccaacatgttgcgacgctgtaacgctgcaggcgtcaggatctacgtagacgccgtgatcaaccacatgacgggcgagcccccggagaacgtgggcactgcaggcagcaccgccgtgttctctgagtggaactaccccgccgtgcccttcaaccggcagcacttcaactggccgcactgcgtcatcaacggcattgactacgctactgacgcttggagagtccgtaactgtgagctcgtgggattgaaggacttgaaccaaggcgacgagcatgtcaggaatatgatcgtcaacttcatgaacgaactcatcgacttgggtgttgctggattcaggatcgacgcagccaaacacatgtggcccgctgacctccgcatcatctacgatcgtctccacaacctcaacacagcccacggcttcccagccaacgcccgtccttacatctaccaagaagtcattgactacggcggcgaggccattagcagagacgaatacacccccataggagctgtgacagagttcaaagtgggtttggaactgagtaacgcgttcagagggaataaccagctccgttggttatcaacttggggtcctcaatggggattgttagcccatgaagactctctgaccttcatcgataaccacgacaatgagagaggacacggaggtggtggtggtatccttacctacaaagagtctcgtccctacaagggtgctatcgccttcctacttgcccatccgtacggagagccgcaaatcatgagcagtttcgacttctgggacagcgaagttggtcctccaatggataacaatggcaacatcatttcacctagtatcaattctgatggaacctgtggtaacggctggatatgccagcaccgttggcggcagatctactccatggtggcgttcaggaacgcggccggtagcactggtatcaacgactggtgggacaatggcgccaaccagatcgctttctgtcgcggaggcaacggtttcgttgccttcaataatgattattgggatcttaaccagaatctacagacatgcttgcccgcaggcagatattgtgatgtgatatctggtgagaaggtgggcagttcttgcacgggcaagactgtcaccgtgggcagtgacggacgggcacacatcagtgtgggagccaacgagtttgatatgatgctcgccattcatgttgggcctgagagtcgtctttaagatggaagctggtcaagtaataagaacaataaaaatgaatttataagtgaattctcatttatcttccaaagtgccttacaaaacacctttgtaaaataaatatcaataaacaacattaaaaaaaa

SEQ ID NO: 23 - DNA, 908 bp, Spodoptera frugiperda
  source 1..908: mol_type = other DNA
gatactatctattcccattgtatatgattacagatctattacaataatggattcagctgtggcgggacactcctgacccagcgacacgtgttgtctgcagcacactgcttcgttactacgaacggagtagtcataagtccctcaacgatcacgattcgcgtcgggtcgactttcctatattcaggtggcgccacacatcaagtatcagcaatcatagtccacgagagctatggtaatccaatcagggacaatgatatagcagtgctactgctcagaacagctgtcaccttgaatgcaagcgtcgctactgcctttatacccacgcaaggatctgttgtgcctgacaacgccaccgtcattgccgtcggctggggggtgactattcaaaatggtcaaaatccttcgcctgtgctcaacgaggtggcaattaggaagatcaaccgtgggatatgccaggccaactacttggagctacaagccttggaaggaattccttatcctgtgaccagcagcatgatctgcgccggcatccttggagttggaggtaaagatgcgtgtcaaggtgattccggtggtcctctgatttacaatgggatagtagtgggggtcacatcatggggatatggatgcgcgcagccgcgataccctggggtgtctgcccgggtcgcaagctacaccacgtggataaacaacacggttacccggtacaatggcgcgagttcagtcggcgtggcgcgagtcactctgctgattcccttcttatatctaatactctctagaagctactgagtaaaccaatcaaagtgacgctagtgtatattttttaagattttcatttttatactgacattgtaatatacgtaaatgcttctcaagagaattaataaaattgccaacttaaccggtgt

SEQ ID NO: 24 - DNA, 1315 bp, Spodoptera frugiperda
  source 1..1315: mol_type = other DNA
gtatcgaacagtcacttggtcgacaacaacaatgcgcgtcatcatcttcctagcccttctgggcaccgctctcgcggtccccaagagcatcagccgtatcgtcggcggcagcccgaccaccgtgcaggaatacccatacatgtccaatatgcagtacggtttttggggcatctggtggttccaggcctgcggtggatctctgctgacctcaacctccgtgctctctgctgctcactgctacttcggtgacagtgcttcggagtggcgtgtgcgcctgggtacctctttcgcgtccagcggtggctctgagcacacagtgtcccagctggtcctgcaccagcagtacaaccccaacaccctcgaccatgatatcgccattgtccgtctggccaaccctgccgtatactccaacagcgtccagcccgctagcgtcgccggcagcgcttacaatcttcctgacaacaccctcgtcaccaccatcggatggggaacaacctcgagcggcggctcttcccccgagcaactgcagcacgtggacatcaacatcatcaaccagcagctgtgtgctgagcgctacctctttcgcgtccagcggtggctctgagcacacagtgtcccagctggtcctgcaccagcagtacaaccccaacaccctcgaccatgatatcgccattgtccgtctggccaaccctgccgtatactccaacagcgtccagcccgctagcgtcgccggcagcgcttacaatcttcctgacaacaccctcgtcaccaccatcggatggggaacaacctcgagcggcggctcttcccctgagcaactgcagcacgtggacatcaacatcatcaaccagcagctgtgtgctgagcgctacgcctacctgaagacccagcccggctaccaaaactggcctgacatcaacatcatcaaccagcagctgtgtgctgagcgctacgcctacctgaagacccagcccggctaccagaactggcctgacatcaccgacaacatgctgtgcgccggtatcttaaacgtcggaggaaaggacgcctgccagggagactctggcggtcccctcgctcacaacaagaatgtcatcgttggtgtcgtctcctggggcttccagtgcgctgatgctttctaccctggtgtcaacgctcgcgtctcccaatacacccaatggatctcccagaacgcctaaataataagtaggcattgtttgtcatcccatgtaaataagaagtctgataaatgttatgtcaaatcaactacctactgaaacacaatacgggtttcgcaaaataaactataatttta

SEQ ID NO: 25 - DNA, 990 bp, Spodoptera frugiperda
  source 1..990: mol_type = other DNA
ggtataaattgtcaattagtttcaacaattaacagtgggtgtagcagctgtgaagctttcatcatgatttctttcttcatctatttggcttttgttagttatgtcgtgtcaagccccacgataaaatccctagaggatcttaaaatcgttggaggtgagaatatcgaaataactgatgcaccgtaccaggtgtctcttgtcagaagggggagacactcctgtggaggaactatagtcgctaaggatattattgtcactgctgctcattgtgtcatgggatcaatggcaaatgaattccaaatccgtgctggttcatcttatagcgagaagggaggacagctatacccagtgggagagattctctggcatccaagctttgactatggaaaaatggacagtgacattgccatactgtggctgcccaaaccgctagtgttcagtgacaaaataagaccagtacaattaatgggacgaggtgaagaaataaaggatggtgatgatacgattgtgactggatggggtaatctttatgaaattggaggcacgccaagcactctgcagagggttacagttcagaaaatcaatgaagctatatgcaatgacgcttacaaacctttgtatgccatcactaatcacatgctctgtgctggagcacctggtggtggaaaggacgcttgccagggtgacagcggcggacctctcgttcacaacaataagttagctggtgtcgtatcctggggtctaggatgtgcccgacctgaataccctggagtctactccaaggtctcagcattgaggagctggctcgatgataacatctatcaactcaggtggaaacatatctggagaagtatttaaaagacaagaagacgaatagacgagaagactagaagaatgagtttggtcttttcgtgcgtgtcagaacggttgtgatttaatgattgattaagttatatgtattgttgtgttcgaattaaattaagacaattg

SEQ ID NO: 26 - DNA, 857 bp, Spodoptera frugiperda
  source 1..857: mol_type = other DNA
tcaattcattacgattcgttgacataatggcgaagctagtgtgtgtcttattcttggtggcagcagcctgcgccaatgcggtccccaaccgcatcgtggggggcaaccccaccgtgatcgagaactaccccagcatcgtgcaggtggagagccgcaccggcatcatctggggacagtcgtgtgctgctagcatcctcaacaccagattcgtagtgtctgctgcgcactgctttgctggaatcctcaacacccgcaaccgtcgtatcagagccggtacctcaaaccgtgcgtctggtggtgtcgtggtctctatcaaccgcatcgtcaaccaccccacttatggcaacaaccagatggacggtgacatcagcgtcatccagctggcttcagccctggtctacaacgaccgcatccaacaagtccccattgtgcctcaagggtttgagctccctgataacctgcctgttgtgcattctggatggggtgccacttctcaaggcggtctggcctcagcaatccttcgcgacgttacgatctacaccatcaaccacgacatctgcgcttctcgctacgccagccttctcttccccagaagagttactcaaaacatgatctgcgccggtatccttgacgtcggtggcaaggacgcctgccagggtgactctggtggtcctctctattacgatggcatcctggtcggtatcgtctcctggggccgtggatgtgctgatcccgtctaccctggagtcagcaccaaggtcgcttcctacaccgactggatcgtacaaactgtttcctctctgtaaattattttatttataggctctgatattgatattataagtgttatgttttga

SEQ ID NO: 27 - DNA, 889 bp, Spodoptera frugiperda
  source 1..889: mol_type = other DNA
cacagttcttctatttcttgcatgtttaacatgcgtgttattattcttttagctcttttgggtgcagcgcttgctgcgcccaacaacccgagccgcatagttggtggctcagccactaccatccaaaattacccttacatgtccaacatgcagttcctttggtgggatttcttctgggcacagacatgtggaggatccttgctcactccttactcagtactctcggcagcacactgcttctatggtgacgaagcctcgcaatggcgtgtagtcctcggaagcacatggggaaacagtggcggcattcctcacccggtcagccagctagtgttacacccacaatacgatcgtgtcactttggaccatgacatcgccattgtacgcctcagcaggcccgccgtgtattctgacatagtccaacctgccagtatcgctggcgctaattataacctcgccgatggcaccgtcgtcgaagttattggatggggcgcaatttggcagctgtgtgctgagcgctacgcctacctgaagacccagcccggctaccaaaactggcctgacatcaccgacaacatgctgtgcgccggtatcttaaacgtcggaggcaaggacgcctgccagggagactctggcggtcccctcgctcacaaccacaatgtcatcgttggtgttgtctcttggggttacgaatgcgctgattcattctaccctggtgtcaatgttcgtgtttcaagatatgcaagctggatttcagccaacgcataaatttatattcgattaagaataaaggatactgaaatgaatgaactgttcctatctgcagtttatttatttatgtcgcaattatagatctttaactgaatacttattaaattagatttaagtaatg

SEQ ID NO: 28 - DNA, 973 bp, Spodoptera frugiperda
  source 1..973: mol_type = other DNA
aaagaacgcggctcgtcttgaccattgtatcgaacagtcacttggtcgacaacaacaatgcgcgtcatcatcttcctagcccttctgggcaccgctctcgcggtccccaagagcatcagccgtatcgtcggcggcagcccgaccaccgtgcaggaatacccatacatgtccaatatgcagtacggtttttggggcatctggtggttccaggcctgcggtggatctctgctgacctcaacctccgtgctctctgctgctcactgctacttcggtgacagtgcttcggagtggcgtgtgcgcctgggtacctctttcgcgtccagcggtggctctgagcacacagtgtcccagctggtcctgcaccagcagtacaaccccaacaccctcgaccatgatatcgccattgtccgtctggccaaccctgccgtatactccaacagcgtccagcccgctagcgtcgccggcagcgcttacaatcttcctgacaacaccctcgtcaccaccatcggatggggaacaacctcgagcggcggctcttcccccgagcaactgcagcacgtggacatcaacatcatcaaccagcagctgtgtgctgagcgctacgcctacctgaagacccagcccggctaccaaaactggcctgacatcaccgacaacatgctctgcgctggtatcttaaacgtcggaggcaaggacgcctgccagggagactctggcggccccctcgctcacaacaagaatgtcatcgttggtgttgtctcctggggcttccagtgcgctgatgctttctaccctggtgtcaacgctcgcgtctcccaatacacccaatggatctcccagaacgcctaaataataagcattatttgtcatcccatgtaaataagaagtctgataaacgttatgtcaactcaactattgaaacacaatacgggtttcgcaaaataaactataattttaaggcttaaagattgtt

(19) United States
(12) Patent Application Publication
(10) Pub. No.: US 2026/0168020 A1
(43) Pub. Date: Jun. 18, 2026

(54) SINGLE CELL SEQUENCING AND USES THEREOF FOR PEST CONTROL

(71) Applicant: Syngenta Crop Protection AG, Basel (CH)

(72) Inventors: Jun Zhang, Research Triangle Park, NC (US); Md Shamimuzzaman, Lincoln, NE (US)

(73) Assignee: Syngenta Crop Protection AG, Basel (CH)

(21) Appl. No.: 19/394,017

(22) Filed: Nov. 19, 2025

Related U.S. Application Data
(60) Provisional application No. 63/722,815, filed on Nov. 20, 2024.

(51) Int. Cl. C12Q 1/6869; C40B 30/00; C40B 40/08; C40B 50/06

(52) U.S. Cl. CPC C12Q 1/6869; C40B 30/00; C40B 40/08; C40B 50/06; C12Q 2600/124; C12Q 2600/158

(57) ABSTRACT

Provided herein are methods for identifying altered expression profiles in a population of plant pests that have developed resistance to a pesticidal protein. The methods comprise isolating a population of cells from each of a resistant and a susceptible plant population, and generating a transcriptome library from each population by performing single cell sequencing analysis on the population of cells. The transcripts are clustered using genetic markers associated with a single cell type and these cell type specific transcripts are compared to identify any transcripts that have an altered expression pattern between the resistant and susceptible populations.

### CROSS REFERENCE TO RELATED APPLICATION

This application claims priority to U.S. Provisional Application No. 63/722,815, filed on Nov. 20, 2024, the contents of which are incorporated herein by reference in its entirety.

### FIELD OF THE INVENTION

The present disclosure relates to methods for identifying target receptors of pesticidal proteins having insecticidal activity.

### STATEMENT REGARDING ELECTRONIC SUBMISSION OF A SEQUENCE LISTING

A Sequence Listing in XML format, submitted under 37 C.F.R. § 1.831-1.835, entitled PAT-109891-US-UTL-1_ST26.xml, generated on Nov. 14, 2025, and approximately 75 KB in size, has been filed via EFS-Web and is provided in lieu of a paper copy. This Sequence Listing is hereby incorporated by reference into the specification in its entirety.

### BACKGROUND

Insects are a major cause of crop losses. Numerous commercially valuable plants, including common agricultural crops, are susceptible to attack by plant pests including insects and nematodes, causing substantial reductions in crop yield and quality. For example, plant pests are a major factor in the loss of the world's important agricultural crops. Insect pests are also a burden to vegetable and fruit growers, to producers of ornamental flowers, and they are a nuisance to home gardeners.

Biological pest control agents, such as Bacillus thuringiensis (Bt) strains expressing pesticidal toxins like δ-endotoxins (delta-endotoxins; also called crystal toxins or Cry proteins), have been applied to crop plants with satisfactory results against insect pests. The δ-endotoxins are proteins held within a crystalline matrix that are known to possess insecticidal activity when ingested by certain orders and species of plant pests, including insects, but are harmless to plants and other non-target organisms. Several native Cry proteins from Bacillus thuringiensis, or engineered Cry proteins, have been expressed in transgenic crop plants to control certain Lepidopteran and Coleopteran insect pests as an alternative to or complement to chemical pesticides. Transgenic corn hybrids that control corn rootworm have been available commercially in the US since 2003 and express toxins such as Cry3Bb1, Cry34Ab1/Cry35Ab1, modified Cry3A (mCry3A), or Cry3Ab (eCry3.1Ab).

Although the usage of transgenic plants expressing Cry proteins has been shown to be extremely effective, insect pests that now have resistance against the Cry proteins expressed in certain transgenic plants are known. Therefore, there remains a need to identify the mechanism of resistance, as well as new and effective pest control agents useful against the resistant species. Particularly needed are proteins that are toxic to pest species of commercially-relevant plants that have a different mode of action than existing insect control products as a way to mitigate the development of resistance.

### SUMMARY

Provided herein are methods for identifying altered expression profiles in a population of plant pests that have developed resistance to a pesticidal protein. The methods comprise isolating a population of cells from each of a resistant and a susceptible plant population, and generating a transcriptome library from each population by performing single cell sequencing analysis on the population of cells. The transcripts are clustered using genetic markers associated with a single cell type and these cell type specific transcripts are compared to identify any transcripts that have an altered expression pattern between the resistant and susceptible populations. In various embodiments, the isolated population of cells consists essentially of primary midgut cells, for example, columnar, goblet, or stem cells. In certain embodiments, the plant pest is a lepidopteran, coleopteran, hemipteran, or dipteran pest and/or the transcript having an altered expression pattern encodes a receptor protein, for example, ATP-binding cassette (ABC) transporter, aminopeptidase N, cadherin, alkaline phosphatase, chitinase and/or lipase families of receptors. In some embodiments, the transcript that has an altered expression pattern or profile in a resistant plant pest is a transcript that is an allelic variant of the same transcript in the susceptible plant pest. The plant pest may be selected from Plutella spp., Helicoverpa spp., Spodoptera spp., or Ostrinia spp.

Thus, the methods provided herein are useful for elucidating resistant mechanisms by identifying one or more altered receptors that cause or contribute to resistance to a pesticidal agent. The methods may further aid in investigating the structure-function relationships of toxin receptors; investigating toxin-receptor interactions; elucidating the mode of action of toxins; screening and identifying novel toxin receptor ligands including novel insecticidal toxins; designing and developing novel toxin receptor ligands; and creating insects or insect colonies with altered susceptibility to insecticidal toxins. The methods provided herein are also useful for managing toxin resistance in plant pests, for monitoring of toxin resistance in plant pests, and for identifying alternative toxins that are capable of protecting plants against damage by resistant plant pests.

### BRIEF DESCRIPTION OF THE DRAWINGS

FIG. 1 shows R2 reads from single cell sequencing of North American fall armyworm (left panel) and Brazilian fall armyworm (right panel) midgut mapped to the columnar cell marker Sf.BR.2.1.0000305.t2 (SEQ ID NO:5).

### BRIEF DESCRIPTION OF THE SEQUENCES IN THE SEQUENCE LISTING

SEQ ID NO: 1 is a transcript that is orthologous to marker Oatp58Dc derived from Drosophila melanogaster.
SEQ ID NO:2 is a transcript that is orthologous to marker Oatp58Dc derived from Drosophila melanogaster.
SEQ ID NO:3 is a transcript that is orthologous to marker Oatp58Dc derived from Drosophila melanogaster.
SEQ ID NO:4 is a transcript that is orthologous to marker Alp2 derived from Drosophila melanogaster.
SEQ ID NO:5 is a transcript that is orthologous to marker Alp2 derived from Drosophila melanogaster.
SEQ ID NO:6 is a transcript that is orthologous to marker Alp2 derived from Drosophila melanogaster.
SEQ ID NO:7 is a transcript that is orthologous to marker lab derived from Drosophila melanogaster.
SEQ ID NO:8 is a transcript that is orthologous to marker lab derived from Drosophila melanogaster.
SEQ ID NO:9 is a transcript that is orthologous to marker lab derived from Drosophila melanogaster.
SEQ ID NO:10 is a transcript that is orthologous to marker Vha100-4 derived from Drosophila melanogaster.
SEQ ID NO:11 is a transcript that is orthologous to marker Vha100-4 derived from Drosophila melanogaster.
SEQ ID NO: 12 is a transcript that is orthologous to marker Jon99Ciii derived from Drosophila melanogaster.
SEQ ID NO:13 is a transcript that is orthologous to marker Jon99Ciii derived from Drosophila melanogaster.
SEQ ID NO: 14 is a transcript that is orthologous to marker Jon99Ciii derived from Drosophila melanogaster.
SEQ ID NO:15 is a transcript that is orthologous to marker Alp4 derived from Drosophila melanogaster.
SEQ ID NO: 16 is a transcript that is orthologous to marker Alp4 derived from Drosophila melanogaster.
SEQ ID NO:17 is a transcript that is orthologous to marker Alp4 derived from Drosophila melanogaster.
SEQ ID NO:18 is a transcript that is orthologous to marker Smvt derived from Drosophila melanogaster.
SEQ ID NO:19 is a transcript that is orthologous to marker Smvt derived from Drosophila melanogaster.
SEQ ID NO:20 is a transcript that is orthologous to marker Amy-p derived from Drosophila melanogaster.
SEQ ID NO:21 is a transcript that is orthologous to marker Amy-p derived from Drosophila melanogaster.
SEQ ID NO:22 is a transcript that is orthologous to marker Amy-p derived from Drosophila melanogaster.
SEQ ID NO:23 is a transcript that is orthologous to marker lambdaTry derived from Drosophila melanogaster.
SEQ ID NO:24 is a transcript that is orthologous to marker lambda Try derived from Drosophila melanogaster.
SEQ ID NO:25 is a transcript that is orthologous to marker lambdaTry derived from Drosophila melanogaster.
SEQ ID NO:26 is a transcript that is orthologous to marker betaTry A derived from Drosophila melanogaster.
SEQ ID NO:27 is a transcript that is orthologous to marker betaTry A derived from Drosophila melanogaster.
SEQ ID NO:28 is a transcript that is orthologous to marker betaTry B derived from Drosophila melanogaster.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments, any feature or combination of features set forth herein can be excluded or omitted. In addition, numerous variations and additions to the various embodiments suggested herein will be apparent to those skilled in the art in light of the instant disclosure, which do not depart from the instant disclosure. Hence, the following descriptions are intended to illustrate some particular embodiments of the disclosure, and not to exhaustively specify all permutations, combinations, and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

For clarity, certain terms used in the specification are defined and presented as follows:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the word “or” also encompasses “and/or” unless the context clearly indicates otherwise.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this disclosure (e.g., in combinations with temperature or molecular weight values) the exact value (i.e., without “about”) is preferred.

As used herein, phrases such as “between about X and Y”, “between about X and about Y”, “from X to Y” and “from about X to about Y” (and similar phrases) should be interpreted to include X and Y, unless the context indicates otherwise. Units, prefixes and symbols may be denoted in their SI accepted form. Unless otherwise indicated, nucleic acids are written left to right in 5′ to 3′ orientation; amino acid sequences are written left to right in N-terminus to C-terminus orientation, respectively. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Activity” of the insecticidal proteins of the disclosure is meant that the insecticidal proteins function as orally active insect control agents, have a toxic effect (e.g., inhibiting the ability of the insect pest to survive, grow, and/or reproduce), and/or are able to disrupt or deter insect feeding, which may or may not cause death of the insect. When an insecticidal protein of the disclosure is delivered to the insect, the result is typically death of the insect, or the insect does not feed upon the source that makes the insecticidal protein available to the insect. “Pesticidal” is defined as a toxic biological activity capable of controlling a pest, such as an insect, nematode, fungus, bacteria, or virus, preferably by killing or destroying them. “Insecticidal” is defined as a toxic biological activity capable of controlling insects, preferably by killing them. A “pesticidal agent” is an agent that has pesticidal activity, including insecticidal activity. An “insecticidal agent” is an agent that has insecticidal activity.

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample.

Barcodes can allow for identification and/or quantification of individual sequencing reads (e.g., a barcode can be or can include a unique molecular identifier or “UMI”). Barcodes can spatially-resolve molecular components found in biological samples, for example, a barcode can be or can include a “spatial barcode”. In some embodiments, a barcode includes both a UMI and a spatial barcode. In some embodiments the UMI and barcode are separate entities. In some embodiments, a barcode includes two or more sub-barcodes that together function as a single barcode. For example, a polynucleotide barcode can include two or more polynucleotide sequences (e.g., sub-barcodes) that are separated by one or more non barcode sequences.

A “cell type specific” population of cells refers to an isolated or homogeneous population of cells consisting essentially of cells that exhibit similar structure and function (i.e., have similar or the same molecular, morphological, physiological, and functional properties) that are distinct from cells of other types. A “homogeneous” population is a population in which at least about 90%, at least about 95%, 96%, 97%, 98%, 99%, or 100% of the cells in the population are the same cell type. Cell types can be identified using markers that are associated with specific cell types. Cell type specific profiles can be obtained from private or publicly available sources or can be generated using the methods herein for a certain cell type, and then used as the reference profile to compare to the expression profile of other species (e.g., susceptible vs resistant). Exemplary cell type specific profiles include those of Drosophila melanogaster (Corrales et al. (2022) Neural Dev 17:8), Spodoptera spp. (Arya et al. (2024) Genomics 116:110898), and Aedes aegypti (Cui and Franz (2020) Insect Biochem Mol Biol. 127:103496).

A “coding sequence” is a nucleic acid sequence that is transcribed into RNA such as mRNA, rRNA, tRNA, snRNA, sense RNA or antisense RNA which is then preferably translated in an organism to produce a protein.

To “control” insects means to inhibit, through a toxic effect, the ability of insect pests to survive, grow, feed, and/or reproduce, and/or to limit insect-related damage or loss in crop plants and/or to protect the yield potential of a crop when grown in the presence of insect pests. To “control” insects may or may not mean killing the insects, although it preferably means killing the insects. In some embodiments of the disclosure, “control” of the insect means killing the insects.

The terms “comprises”, “comprising, “includes”, “including”, “having” and their conjugates mean including “but not limited to”. These terms specify the presence of stated features, integers, steps, operations, elements, or components, but do not preclude the presence or addition of one or more other features, integers, steps, operations, elements, components, or groups thereof. The term “consisting of means “including and limited to”.

As used herein, the transitional phrase “consisting essentially of” (and grammatical variants) means that the scope of a claim is to be interpreted to encompass the specified materials or steps recited in the claim” and those that do not materially alter the basic and novel characteristic(s)” of the claimed disclosure. Thus, the term “consisting essentially of” when used in a claim of this disclosure is not intended to be interpreted to be equivalent to “comprising.”

In the context of the disclosure, “corresponding to” or “corresponds to” means that when the amino acid sequences of a reference sequence are aligned with a second amino acid sequence (e.g. variant or homologous sequences), different from the reference sequence, the amino acids that “correspond to” certain enumerated positions in the second amino acid sequence are those that align with these positions in the reference amino acid sequence but that are not necessarily in the exact numerical positions relative to the particular reference amino acid sequence of the disclosure.

To “deliver” or “delivering” a composition or an insecticidal protein means that the composition or insecticidal protein comes into contact with an insect, which facilitates the oral ingestion of the composition or insecticidal protein, resulting in a toxic effect and control of the insect. The composition or insecticidal protein may be delivered in many recognized ways, e.g., through a transgenic plant expressing the insecticidal protein, formulated protein composition(s), sprayable protein composition(s), a bait matrix, or any other art-recognized toxin delivery system.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability, or function of a protein. Identified by their high degree of conservation in aligned sequences of a family of protein homologues, they can be used as identifiers to determine if any polypeptide in question belongs to a previously identified polypeptide group.

An “engineered” protein of the disclosure refers to a protein that has a sequence that is different at least one amino acid position compared to at least one corresponding parent protein. An engineered protein can be a mutant protein that contains, e.g., one or more modifications such as deletions, additions, and/or substitutions of one or more amino acid positions relative to a parent protein. An engineered protein can be a chimeric protein and contain, e.g., one or more swapped or shuffled domains or fragments from at least two parent proteins.

“Effective pest-controlling amount” means that concentration of a pesticidal protein that inhibits, through a toxic effect, the ability of pests to survive, grow, feed and/or reproduce, or to limit pest-related damage or loss in crop plants. “Effective pest-controlling amount” may or may not mean killing the pests, although it preferably means killing the pests. A transgenic plant with “enhanced pesticidal properties” is a plant that expresses a protein or proteins at effective pest-controlling amounts, so that, in some embodiments, the plant is pesticidal to an increased range of pest species, relative to a plant of the same kind which is not transformed. This increased range of pest species includes pest plant pests, such as coleopteran, lepidopteran, dipteran, and nematode pests.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

The term “heterologous” when used in reference to a gene or a polynucleotide or a polypeptide refers to a gene or a polynucleotide or a polypeptide that is or contains a part thereof not in its natural environment (i.e., has been altered by the hand of man). For example, a heterologous gene may include a polynucleotide from one species introduced into another species. A heterologous gene may also include a polynucleotide native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the disclosure, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, a “heterologous” polynucleotide refers to a polynucleotide not naturally associated with a host cell into which it is introduced, including non-naturally occurring multiple copies of a naturally occurring polynucleotide. A heterologous nucleic acid sequence or nucleic acid molecule may comprise a chimeric sequence such as a chimeric expression cassette, where the promoter and the coding region are derived from multiple source organisms. The promoter sequence may be a constitutive promoter sequence, a tissue-specific promoter sequence, a chemically-inducible promoter sequence, a wound-inducible promoter sequence, a stress-inducible promoter sequence, or a developmental stage-specific promoter sequence.

A “homologous” nucleic acid sequence is a nucleic acid sequence naturally associated with a host cell into which it is introduced.

The terms “increase”, “increasing”, “increased”, “enhance”, “enhanced”, “enhancing”, and “enhancement” and similar terms, as used herein, describe an elevation in control of a plant pest, e.g., by contacting a plant with a polypeptide of the disclosure (such as, for example, by transgenic expression or by topical application methods). The increase in control can be in reference to the level of control of the plant pest in the absence of the polypeptide of the disclosure (e.g., a plant that is not transgenically expressing the polypeptide or is not topically treated with the polypeptide). Thus in embodiments, the terms “increase”, “increasing”, “increased”, “enhance”, “enhanced”, “enhancing”, and “enhancement” and similar terms can indicate an elevation of at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500% or more as compared to a suitable control (e.g., a plant, plant part, plant cell that is not contacted with the polypeptide of the disclosure).

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned.

Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend-0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47 (W1): W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

“Insecticidal” as used herein is defined as a toxic biological activity capable of controlling an insect pest, optionally but preferably by killing them. The term “pesticidal” more broadly applies to a toxic biological activity capable of controlling insects and other biotic plant pests, such as nematodes or fungi.

In some embodiments, the polynucleotides or polypeptides of the disclosure are “isolated”. The term “isolated” polynucleotide or polypeptide is a polynucleotide or polypeptide that no longer exists in its natural environment. An isolated polynucleotide or polypeptide of the disclosure may exist in a purified form or may exist in a recombinant host such as in a transgenic bacteria or a transgenic plant. Therefore, for example, a claim to an “isolated” polynucleotide or polypeptide encompasses a nucleic acid molecule when the nucleic acid molecule is comprised within a transgenic plant genome.

The term “isolated”, when used in the context of the nucleic acid molecules or polynucleotides of the present disclosure, refers to a polynucleotide that is identified within and isolated/separated from its chromosomal polynucleotide context within the respective source organism. An isolated nucleic acid or polynucleotide is not a nucleic acid as it occurs in its natural context if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In some embodiments, the nucleic acid molecules of the present disclosure are isolated.

A “native” or “wild type” nucleic acid, polynucleotide, nucleotide sequence, polypeptide or amino acid sequence refers to a naturally occurring or endogenous nucleic acid, polynucleotide, nucleotide sequence, polypeptide, or amino acid sequence.

A “nucleic acid molecule” or “nucleic acid” is a segment of single-stranded, double-stranded, or partially double-stranded DNA or RNA, or a hybrid thereof, that can be isolated or synthesized from any source. In the context of the present disclosure, the nucleic acid molecule is typically a segment of DNA. In some embodiments, the nucleic acid molecules of the disclosure are isolated nucleic acid molecules. In some embodiments, the nucleic acid molecules of the disclosure are comprised within a vector, a plant, a plant cell, or a bacterial cell. The terms also include reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A nucleic acid molecule can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically, or metabolically modified forms of polynucleotides, as well as the chemical forms of DNA and RNA characteristic of viruses and cells, including inter alia, simple, and complex cells.

The terms “nucleic acid,” “nucleic acid molecule,” and “polynucleotide” are used interchangeably herein.

An “ortholog” as used herein is a gene that is homologous to genes found in different organisms that share a common ancestry and perform similar biological functions.

As used herein “pesticidal,” insecticidal,” and the like, refer to the ability of proteins of the disclosure to control a pest organism or an amount of one or more proteins of the disclosure that can control a pest organism.

The term “plant” includes reference to whole plants, plant organs, plant tissues (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes, and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

As used herein, “plant material,” “plant part” or “plant tissue” means plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes, and the like. Any tissue of a plant in planta or in culture is included in the term “plant tissue.”

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein “plant sample” or “biological sample” refers to either intact or non-intact (e.g. milled seed or plant tissue, chopped plant tissue, lyophilized tissue) plant tissue. It may also be an extract comprising intact or non-intact seed or plant tissue. The biological sample or extract may be selected from the group consisting of corn flour, corn meal, corn syrup, corn oil, corn starch, and cereals manufactured in whole or in part to contain corn by-products.

A “polynucleotide of interest” or “nucleic acid of interest” refers to any polynucleotide which, when transferred to an organism, e.g., a plant, confers upon the organism a desired characteristic such as insect resistance, disease resistance, herbicide tolerance, antibiotic resistance, improved nutritional value, improved performance in an industrial process, production of a commercially valuable enzyme or metabolite, an altered reproductive capability, and the like.

A “portion” or a “fragment” of a polypeptide of the disclosure will be understood to mean an amino acid sequence or nucleic acid sequence of reduced length relative to a reference amino acid sequence or nucleic acid sequence of the disclosure. Such a portion or a fragment according to the disclosure may be, where appropriate, included in a larger polypeptide or nucleic acid of which it is a constituent (e.g., a tagged or fusion protein or an expression cassette). In embodiments, the “portion” or “fragment” substantially retains the activity, such as insecticidal activity (e.g., at least 40%, 50%, 60%, 70%, 80%, 85%, 90%, 95% or even 100% of the activity) of the full-length protein or nucleic acid, or has even greater activity, e.g., insecticidal activity, than the full-length protein).

The terms “protein,” “peptide,” and “polypeptide” are used interchangeably herein.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule which may be incorporated into its genome. As a result of such genomic alteration, the recombinant plant is distinctly different from the related wild-type plant. A “recombinant” bacteria is a bacteria not found in nature that comprises a heterologous nucleic acid molecule.

Such a bacteria may be created by transforming the bacteria with the nucleic acid molecule or by the conjugation-like transfer of a plasmid from one bacteria strain to another, whereby the plasmid comprises the nucleic acid molecule.

The terms “reduce,” “reduced,” “reducing,” “reduction,” “diminish,” and “suppress” (and grammatical variations thereof) and similar terms, as used herein, refer to a decrease in the survival, growth and/or reproduction of a plant pest, e.g., by contacting a plant with a polypeptide of the disclosure (such as, for example, by transgenic expression or by topical application methods). This decrease in survival, growth and/or reproduction can be in reference to the level observed in the absence of the polypeptide of the disclosure (e.g., a plant that is not transgenically expressing the polypeptide or is not topically treated with the polypeptide). Thus, in embodiments, the terms “reduce,” “reduced,” “reducing,” “reduction,” “diminish,” and “suppress” (and grammatical variations thereof) and similar terms mean a decrease of at least about 5%, 10%, 15%, 20%, 25%, 30%, 35%, 40%, 45%, 50%, 55%, 60%, 65%, 70%, 75%, 80%, 85%, 90%, 95% or more as compared with a plant that is not contacted with a polypeptide of the disclosure (e.g., a plant that is not transgenically expressing the polypeptide or is not topically treated with the polypeptide). In representative embodiments, the reduction results in no or essentially no (i.e., an insignificant amount, e.g., less than about 10%, less than about 5% or even less than about 1%) detectable survival, growth and/or reproduction of the plant pest.

As used herein, a “resistant plant pest” or “resistant insect” refers to a species of plant pests that is capable of feeding and/or reproducing following exposure to a pesticidal agent that previously killed or controlled said species of plant pest. A “susceptible plant pest” refers to a species of plant pests that, when contacting or ingesting a pesticidal agent, is killed or controlled by the specific pesticidal agent. Thus, when comparing a resistant and a susceptible plant pest, the resistant pest, or population thereof, consists essentially of pests that are not killed or controlled by the same pesticidal agent that is capable of killing or controlling a susceptible pest of the same species. Generally, the resistant pest is a genetic variant of the susceptible pest and has developed an evolutionary advantage over the susceptible pest for growing, feeding and/or reproducing after exposure to the pesticidal agent.

“Synthetic” refers to a nucleotide sequence comprising bases or a structural feature(s) that is not present in the natural sequence. For example, an artificial sequence encoding a protein of the disclosure that resembles more closely the G+C content and the normal codon distribution of dicot or monocot plant genes is said to be synthetic.

As used herein, a protein of the disclosure that is “toxic” to a plant pest is meant that the protein functions as an orally active pest control agent to kill the plant pest, or the protein is able to disrupt or deter pest feeding, or causes growth inhibition to the plant pest, both of which may or may not cause death of the pest. When a toxic protein of the disclosure is delivered to a pest or a pest comes into oral contact with the toxic protein, the result is typically death of the pest, or the growth of the pest is slowed, or the pest stops feeding upon the source that makes the toxic protein available to the pest.

The terms “toxin fragment” and “toxin portion” are used interchangeably herein to refer to a fragment or portion of a longer (e.g., full-length) pesticidal protein of the disclosure, where the “toxin fragment” or “toxin portion” retains pesticidal activity. For example, it is known in the art that native Cry proteins are expressed as protoxins that are processed at the N-terminal and C-terminal ends to produce a mature toxin. In embodiments, the “toxin fragment” or “toxin portion” of a chimeric pesticidal protein of the disclosure is truncated at the N-terminus and/or C-terminus. In embodiments, the “toxin fragment” or “toxin portion” is truncated at the N-terminus to remove part or all of the N-terminal peptidyl fragment, and optionally comprises at least about 400, 425, 450, 475, 500, 510, 520, 530, 540, 550, 560, 570, 580 or 590 contiguous amino acids or an amino acid sequence that is substantially identical thereto. Thus, in embodiments, a “toxin fragment” or “toxin portion” of an pesticidal protein is truncated at the N-terminus (e.g., to omit part or all of the peptidyl fragment), for example, an N-terminal truncation of one amino acid or more than one amino acid, e.g., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids. In embodiments, a “toxin fragment” or “toxin portion” of an pesticidal protein is truncated at the C-terminus (e.g., to omit part or all of the protoxin tail), for example, a C-terminal truncation of one amino acid or more than one amino acid, e.g., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 125, 150, 175, 200, 225, 250, 275, 300, 325, 350, 375, 400, 425, 450, 475, 500, 525, 550, 560 or more amino acids. In embodiments, the “toxin fragment” or “toxin portion” comprises domains 1 and 2, and the core domain 3. In embodiments, the “toxin fragment” or “toxin portion” is the mature (i.e., processed) toxin (e.g., Cry toxin).

“Transcriptome” refers to the complete set of RNA transcripts produced by a cell or organism, including messenger RNA (mRNA), ribosomal RNA (rRNA), and transfer RNA (tRNA). It represents the active genes in a cell or tissue at a specific time and can provide insights into gene expression, regulation, and function.

“Transformed” and “transgenic” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term “transgenic plant” includes reference to a plant into which a heterologous nucleic acid molecule has been introduced. Generally, the heterologous nucleic acid sequence is stably integrated within the genome such that the nucleic acid sequence is passed on to successive generations. The heterologous nucleic acid sequence may be integrated into the genome alone or as part of a recombinant expression cassette. “Transgenic” is used herein to include any cell, cell line, callus, tissue, plant part or plant, the genotype of which has been altered by the presence of a heterologous nucleic acid sequence, including those transgenics initially so altered as well as those created by sexual crosses or asexual propagation from the initial transgenic.

### Overview

Provided herein are methods that are useful for identifying a gene, trait, or genetic locus associated resistance of a plant pest to a pesticidal agent. The methods utilize single cell sequencing to identify the target site of insect control traits and can be utilized to discover causative genes that control or contribute to resistance by analyzing gene expression differences from a population of cell type-specific isolated single cells (e.g., intestinal columnar cells).

Cry toxins, utilized in insecticidal applications, operate through two primary mechanisms: the sequential binding model and the signaling pathway model. The sequential binding model involves a multi-step process where activated Cry toxins bind to various receptors on the mid-intestinal epithelial cell membrane, forming pores that lead to cell lysis and insect death. This process includes crystal dissolution, protoxin activation, receptor binding, oligomer formation, and membrane insertion. Conversely, the signaling pathway model proposes that Cry toxins bind to cadherin receptors, triggering cell signaling cascades that result in cellular apoptosis rather than pore formation. With the widespread use of Bt preparations and transgenic crops, insect resistance to Cry toxins has become an increasing concern, often arising from mutations in receptor genes that weaken toxin-receptor binding. To address this challenge, researchers are employing high-throughput sequencing and multiomics techniques to investigate resistance mechanisms, artificially selecting resistant insects for study, and exploring genetic engineering and synthetic biology approaches to enhance Cry toxin efficacy. Additionally, CRISPR/Cas9 technology is being considered to restore resistant mutants to wild type, while strategies leveraging insect mating and reproduction characteristics are being developed to reduce resistant populations. Understanding these mechanisms and developing novel strategies is crucial for maintaining the effectiveness of Cry toxins in pest control applications.

### Single-Cell Analysis Techniques

Single-cell RNA sequencing (scRNA-seq) is a powerful technology that enables the determination of precise gene expression patterns from tens of thousands of cells. This technology offers an unbiased approach to understand cell type diversity and distinct function from a heterogenous cell population. It has been applied to discover biological variation hidden in sequencing results of pool of different cell types. scRNA-seq technologies useful in the present invention include, but are not limited to:

### 1. Drop-Seq

Drop-Seq analyzes mRNA transcripts from droplets of individual cells in a highly parallel fashion. This single-cell sequencing method uses a microfluidic device to compartmentalize droplets containing a single cell, lysis buffer, and a microbead covered with barcoded primers. Each primer contains: 1) a 30 bp oligo (dT) sequence to bind mRNAs; 2) an 8 bp molecular index to identify each mRNA strand uniquely; 3) a 12 bp barcode unique to each cell and 4) a universal sequence identical across all beads. Following compartmentalization, cells in the droplets are lysed and the released mRNA hybridizes to the oligo (dT) tract of the primer beads. Next, all droplets are pooled and broken to release the beads within. After the beads are isolated, they are reverse-transcribed with template switching. This generates the first cDNA strand with a PCR primer sequence in place of the universal sequence. cDNAs are PCR-amplified, and sequencing adapters are added using the Nextera XT Library Preparation Kit. The barcoded mRNA samples are ready for sequencing. This method is further described in Macosko, Evan Z., et al., Cell, 2015. 161 (5): p. 1202-1214, which is herein incorporated by reference.

2. inDrop

inDrop is used for high-throughput single-cell labeling. This approach is similar to Drop-seq, but it uses hydrogel microspheres to introduce the oligonucleotides. Single cells from a cell suspension are isolated into droplets containing lysis buffer. After cell lysis, cell droplets are fused with a hydrogel microsphere containing cell-specific barcodes and another droplet with enzymes for RT. Droplets from all the wells are pooled and subjected to isothermal reactions for RT. The barcodes anneal to poly(A)+ mRNAs and act as primers for reverse transcriptase. Now that each mRNA strand has cell-specific barcodes, the droplets are pooled and broken, and the cDNA is purified. The 3′ ends of the cDNA strands are ligated to adapters, amplified, annealed to indexed primers, and amplified further before sequencing. This method is further described in Klein, Allon M., et ah, Cell, 2015. 161 (5): p. 1187-1201, which is herein incorporated by reference.

### 3. CEL-Seq

CEL-Seq uses barcoding and pooling of RNA to overcome challenges from low input. In this method, each cell undergoes RT with a unique barcoded primer in its individual tube. After second-strand synthesis, cDNAs from all reaction tubes are pooled and PCR-amplified. Paired-end deep sequencing of the PCR products allows for accurate detection of sequence information derived from both strands. This method, and related CEL-seq2 are further described in Hashimshony, T., et ah, Cell Reports, 2012. 2 (3): p. 666-673 and Hashimshony, T., et ah, Genome Biology, 2016. 17 (1): p. 77, which are herein incorporated by reference.

### 4. Quartz-Seq

The Quartz-Seq method optimizes whole-transcript amplification (WTA) of single cells. In this method, an RT primer with a T7 promoter and PCR target is first added to the extracted mRNA. RT synthesizes first-strand cDNA, after which the RT primer is digested by exonuclease I. Next, a poly(A) tail is added to the 3′ ends of first-strand cDNA, along with a poly(dT) primer containing a PCR target. After second-strand generation, a blocking primer is added to ensure PCR enrichment in sufficient quantity for sequencing. Deep sequencing allows for accurate, high-resolution representation of the whole transcriptome of a single cell.

### 5. MARS-Seq

MARS-Seq profiles the transcriptional dynamics of single cells in an automated and massively parallel workflow with high resolution. MARS-Seq can be used with in vivo samples containing a wide variety of different cell subpopulations. Single cells are first isolated into individual wells using FACS. Each cell is lysed, and the 3′ ends of mRNAs are annealed to unique molecular identifiers containing a T7 promoter. The mRNA is reverse-transcribed to generate the first cDNA strand and treated with exonuclease I to remove leftover RT primers. Next, the cellular lysates are pooled together and converted to double-stranded cDNA. The DNA strands are transcribed to RNA and treated with DNase to remove leftover DNA templates in the mixture. The RNA strands are fragmented and annealed to sequencing adapters, followed by RT to generate barcoded cDNA libraries that are ready for sequencing.

### 6. CytoSeq

CytoSeq enables gene expression profiling of thousands of single cells. In this method, single cells are randomly deposited into wells. A combinatorial library of beads with specific capture probes is added to each well. After cell lysis, mRNAs hybridize the to beads, which are pooled subsequently for RT, amplification, and sequencing. Deep sequencing provides accurate, high-coverage gene expression profiles of several single cells.

### 7. Hi-SCL

Hi-SCL generates transcriptome profiles for thousands of single cells using a custom microfluidics system, similar to Drop-Seq and inDrop. Single cells from cell suspension are isolated into droplets containing lysis buffer. After cell lysis, cell droplets are fused with a droplet containing cell-specific barcodes and another droplet with enzymes for RT. The droplets from all the wells are pooled and subjected to isothermal reactions for RT. The barcodes anneal to poly(A)+ mRNAs and act as primers for reverse transcriptase. Now that each mRNA strand has cell-specific barcodes, the droplets are broken, and the cDNA is purified. The 3′ ends of the cDNA strands are ligated to adapters, amplified, annealed to indexed primers, and amplified further before sequencing.

### 8. Seq-Well

Single-cell RNA-seq can precisely resolve cellular states, but applying this method to low-input samples is challenging. Here, the inventors present Seq-Well, a portable, low-cost platform for massively parallel single-cell RNA-seq. Barcoded mRNA capture beads and single cells are sealed in an array of subnanoliter wells using a semipermeable membrane, enabling efficient cell lysis and transcript capture. This method is further described in Gierahn et ah, Nat Methods. 2017 April; 14 (4): 395-398, which is herein incorporated by reference. This method is further described in Gierahn, T. M., et al., Nature Methods, 2017. 14: p. 395, which is herein incorporated by reference.

### 9. Microwell-Seq

Microwell-seq confines single cells and barcoded poly(dT) mRNA capture beads in a PDMS array of subnanoliter wells. Well dimensions are designed to accommodate only one bead. Cells are loaded by gravity with a rate of dual occupancy that can be tuned by adjusting the number of cells and loaded and visualized prior to processing. This method is further described in Han, X., et al., Cell, 2018. 172 (5): p.1091-1107.e17, which is herein incorporated by reference.

### 10. Nanogrid-Seq

Nanogrid-seq is a nanogrid platform and microfluidic depositing system that enables imaging, selection, and sequencing of thousands of single cells or nuclei in parallel. This method is further described in Gao, R., et al., Nature Communications, 2017. 8 (1): p. 228, which is herein incorporated by reference.

### 11. SCI-Seq

Sci-seq refers to Single cell Combinatorial Indexed Sequencing (SCI-seq) that can be used as a means of simultaneously generating thousands of low-pass single cell libraries for somatic copy number variant detection. This is further described in Vitak, S. A., et al., Nature Methods, 2017. 14: p. 302, which is herein incorporated by reference.

### 12. Direct-Tagmentation

Enzymes called transposases randomly cut the DNA into short segments (“tags”). Adapters are added on either side of the cut points (ligation). Strands that fail to have adapters ligated are washed away. The adaptors may contain barcodes and/or primer binding sites for detection and amplification of the genomic sequences. This is further described in Zahn, H., et al., Nature Methods, 2017. 14: p. 167, which is herein incorporated by reference. 1

13. sciATAC-Seq

sci-ATAC-seq is a single-cell ATAC-seq protocol. This technique can be used to determine chromatin accessibility both between and within populations of single cells. Single cell ATAC-Seq relies on combinatorial cellular indexing, and thus does not require the physical isolation of individual cells during library construction. The technique scales sublinearly in time and cost and can profile thousands of individual cells in a single experiment. This method is further described in Cusanovich, D. A., et al., Science, 2015. 348 (6237): p. 910, which is herein incorporated by reference. A related method, nano-well scATAC-seq is described in Mezger, A., et al., High-throughput chromatin accessibility profiling at single-cell resolution, bioRxiv, 2018, which is incorporated by reference.

### 14. 10× Chromium

The 10× Chromium single cell platform (10× Genomics, Pleasanton, CA) is a technology that uses microfluidics to isolate individual cells and barcode their RNA transcripts, allowing for the simultaneous analysis of thousands of cells in a single experiment. In this method, microfluidics is used to create oil-encapsulated droplets (GEMs-Gel Beads in Emulsion) that each contain millions of oligonucleotides that have a unique 10× barcode sequence, along with oligo-dTs to capture mRNAs, a unique molecular identifier, and the reagents necessary for cell lysis and reverse transcription. Inside each GEM, the cell is lysed, releasing its mRNA which then binds to the oligo-dT sequences on the gel bead. Each mRNA is reverse transcribed, with the resulting cDNA each having the cell's unique barcode, the GEM's unique molecular identifier, and the gene-specific sequence. Following reverse transcription, the beads are lysed and all barcoded cDNA is pooled for amplification via PCR. The resulting library is sequenced using standard next-gen sequencing (e.g., Illumina). Bioinformatics is used to analyze the gene expression patterns.

Various other scRNA-seq platforms are useful in the present invention, such as, but not limited to: CELseq [Hashimshony et al. (2012) Cell Rep. 2:666-673]; SMART-seq (Ramskold et al. (2012) Nature Biotechnology 30:77]; SMART-seq2 [Picelli et al. (2013) Nat. Methods 10:1096-1098]; 10× Genomics [Zheng et al. (2017) Nat. Commun. 8:14049]; Sci-RNA-seq [Cao et al. (2017) Science 357:661-667]; MATQ-seq [Sheng et al. (2017) Nat. Methods 14:267-270]; SPLIT-seq [Rosenberg et al. (2018) Science 360:176-182].

### Sequencing Methods

The methods of the invention may employ any number of available sequencing technologies such as, but not limited to: Illumina (Solexa) sequencing, including the HiSeq2000, HiSeq2500 and MiSeq systems (Illumina, San Diego, CA); Roche 454 pyrosequencing (Creative Biogene, Shirley, NY); SOLID sequencing and Ion Torrent semiconductor sequencing (Thermo Fisher Scientific, Carlsbad, CA); DNA nanoball sequencing (Roche Sequencing, Indianapolis, IN); HeliScope single molecule sequencing (Thomson and Steinmann (2010) Curr Protoc Mol Biol, Chapter 7; available on the worldwide web at//europepmc.org/backend/ptpmcrender.fcgi?accid=PMC2954431&blobtype=pdf); and single molecule real time (SMRT) sequencing (Pacific Biosciences, Menlo Park, CA).

### Plant Pests and Resistance Loci

In various embodiments, the present invention provides methods for identifying a gene, trait or genetic loci that is altered (e.g., inserted, deleted, or changed) in a plant pest that has developed resistance to a pesticidal agent relative to a susceptible pest of the same species. By identifying a gene, trait or genetic loci (e.g., allele or alleles) that are modified in the resistant pest, candidate pesticidal compositions can be identified or engineered to control the resistant pest, e.g., by utilizing a different mode of action (the mechanism by which the pesticide impacts the plant pest) or site of action (the physical location within a plant pest where the pesticidal composition acts, i.e., the “target site”) than the pesticidal agent against which the pest has gained resistance.

The insecticidal mechanisms of Cry toxins are diverse and complex, often involving multiple factors (reviewed in Liu et al. (2021) Front. Microbiol. 12:665101). While not fully understood, two main models have been proposed: the sequential binding model and the signaling pathway model. The sequential binding model involves a complex multi-step process where Cry toxins bind to various midgut epithelial cell membrane receptors in a specific order. This binding causes the toxins to mature and form oligomers that insert into the cell membrane, creating pores that lead to osmotic imbalance, cell lysis, and ultimately insect death. The process begins with the dissolution of parasporal crystals in the insect's midgut, followed by protoxin cleavage, binding to receptors like APN, ALP, and cadherin, conformational changes, and oligomer formation. The pores formed allow ions and bacteria to enter the hemocoel, causing sepsis. ABC transporters and other intracellular proteins may also play roles in this process.

The signaling pathway model, on the other hand, proposes that cell death occurs through cellular apoptosis mediated by cadherin receptors, rather than pore formation. In this model, Cry toxin binding to cadherin receptors triggers Mg2+-dependent cell signaling cascades, activating G proteins and adenylate cyclase, increasing cAMP levels, and activating protein kinases A. This leads to disruption of ion channels and cytoskeletons, ultimately causing cell apoptosis and insect death. This model does not involve interactions with other protein receptors or pore formation, simplifying the process and explaining why some insects can be killed by Cry toxins when only cadherin receptors are present.

While resistance to Bt and Cry toxins can arise through various mechanisms that disrupt the insecticidal process, the primary mechanism involves mutations in receptor genes, which reduce the binding affinity between toxins and their target receptors. Insects can evolve mutations in genes encoding receptors that bind Cry toxins, such as Cadherins and cadherin-like proteins, alkaline phosphatase (ALP) proteins, aminopeptidase N (APN) proteins, ATP-binding cassette (ABC) transporter proteins such as ABCA1, ABCA2, ABCB1, ABCC2 and ABCC3. These mutations can reduce or prevent toxin binding, conferring resistance (Bel et al. (2020) Toxins (Basel). 12, 785). Thus, in certain embodiments, the methods disclosed herein are useful for identifying variations in expression patterns between susceptible and resistant plant pests in one or more of a cadherin, cadherin-like protein, an ALP protein, an APN protein, and an ABC transporter protein.

In additional embodiments, the resistant plant pest has developed resistance to the pesticidal activity of one or more of any number of Bacillus thuringiensis pesticidal proteins including, but not limited to, a Cry protein, a vegetative pesticidal protein (VIP) and pesticidal chimeras of any of the preceding pesticidal proteins. In other embodiments, the resistant plant pest has developed resistance to the pesticidal activity of a Cry protein selected from: Cry1Aa, Cry1Ab, Cry1Ac, Cry1Ad, Cry1Ae, Cry1Af, Cry1Ag, Cry1Ah, Cry1Ai, Cry1Aj, Cry1Ba, Cry1Bb, Cry1Bc, Cry1Bd, Cry1Be, Cry1Bf, Cry1Bg, Cry1Bh, Cry1Bi, Cry1Ca, Cry1Cb, Cry1Da, Cry1Db, Cry1Dc, Cry1Dd, Cry1Ea, Cry1Eb, Cry1Fa, Cry1Fb, Cry1Ga, Cry1Gb, Cry1Gc, Cry1Ha, Cry1Hb, Cry1Hc, Cry1la, Cry1Ib, Cry1Ic, Cry1Id, Cry1le, Cry1If, Cry1Ig, Cry1Ja, Cry1Jb, Cry1Jc, Cry1Jd, Cry1Ka, Cry1La, Cry1Ma, Cry1Na, Cry1Nb, Cry2Aa, Cry2Ab, Cry2Ac, Cry2Ad, Cry2Ae, Cry2Af, Cry2Ag, Cry2Ah, Cry2Ai, Cry2Aj, Cry2Ak, Cry2Al, Cry2Ba, Cry3Aa, Cry3Ba, Cry3Bb, Cry3Ca, Cry4Aa, Cry4Ba, Cry4Ca, Cry4Cb, Cry4Cc, Cry5Aa, Cry5Ab, Cry5Ac, Cry5Ad, Cry5Ba, Cry5Ca, Cry5 Da, Cry5Ea, Cry6Aa, Cry6Ba, Cry7Aa, Cry7Ab, Cry7Ac, Cry7Ba, Cry7Bb, Cry7Ca, Cry7Cb, Cry7 Da, Cry7Ea, Cry7Fa, Cry7Fb, Cry7Ga, Cry7Gb, Cry7Gc, Cry7Gd, Cry7Ha, Cry7Ia, Cry7Ja, Cry7Ka, Cry7Kb, Cry7La, Cry8Aa, Cry8Ab, Cry8Ac, Cry8Ad, Cry8Ba, Cry8Bb, Cry8Bc, Cry8Ca, Cry8 Da, Cry8Db, Cry8Ea, Cry8Fa, Cry8Ga, Cry8Ha, Cry8Ia, Cry8Ib, Cry8Ja, Cry8Ka, Cry8Kb, Cry8La, Cry8Ma, Cry8Na, Cry8 Pa, Cry8Qa, Cry8Ra, Cry8Sa, Cry8Ta, Cry9Aa, Cry9Ba, Cry9Bb, Cry9Ca, Cry9 Da, Cry9Db, Cry9Dc, Cry9Ea, Cry9Eb, Cry9Ec, Cry9Ed, Cry9Ee, Cry9Fa, Cry9Ga, Cry10Aa, Cry11Aa, Cry11Ba, Cry11Bb, Cry12Aa, Cry13Aa, Cry14Aa, Cry14Ab, Cry15Aa, Cry16Aa, Cry17Aa, Cry18Aa, Cry18Ba, Cry18Ca, Cry19Aa, Cry19Ba, Cry19Ca, Cry20Aa, Cry20Ba, Cry21Aa, Cry21Ba, Cry21Ca, Cry21 Da, Cry21Ea, Cry21Fa, Cry21Ga, Cry21Ha, Cry22Aa, Cry22Ab, Cry22Ba, Cry22Bb, Cry23Aa, Cry24Aa, Cry24Ba, Cry24Ca, Cry25Aa, Cry26Aa, Cry27Aa, Cry28Aa, Cry29Aa, Cry29Ba, Cry30Aa, Cry30Ba, Cry30Ca, Cry30 Da, Cry30Db, Cry30Ea, Cry30Fa, Cry30Ga, Cry31Aa, Cry31Ab, Cry31Ac, Cry31Ad, Cry32Aa, Cry32Ab, Cry32Ba, Cry32Ca, Cry32Cb, Cry32 Da, Cry32Ea, Cry32Eb, Cry32Fa, Cry32Ga, Cry32Ha, Cry32Hb, Cry32Ia, Cry32Ja, Cry32Ka, Cry32La, Cry32Ma, Cry32 Mb, Cry32Na, Cry320a, Cry32 Pa, Cry32Qa, Cry32Ra, Cry32Sa, Cry32Ta, Cry32Ua, Cry33Aa, Cry34Aa, Cry34Ab, Cry34Ac, Cry34Ba, Cry35Aa, Cry35Ab, Cry35Ac, Cry35Ba, Cry36Aa, Cry37Aa, Cry38Aa, Cry39Aa, Cry40Aa, Cry40Ba, Cry40Ca, Cry40 Da, Cry41Aa, Cry41Ab, Cry41Ba, Cry42Aa, Cry43Aa, Cry43Ba, Cry43Ca, Cry43Cb, Cry43Cc, Cry44Aa, Cry45Aa, Cry46Aa Cry46Ab, Cry47Aa, Cry48Aa, Cry48Ab, Cry49Aa, Cry49Ab, Cry50Aa, Cry50Ba, Cry51Aa, Cry52Aa, Cry52Ba, Cry53Aa, Cry53Ab, Cry54Aa, Cry54Ab, Cry54Ba, Cry55Aa, Cry56Aa, Cry57Aa, Cry57Ab, Cry58Aa, Cry59Aa, Cry59Ba, Cry60Aa, Cry60Ba, Cry61Aa, Cry62Aa, Cry63Aa, Cry64Aa, Cry65Aa, Cry66Aa, Cry67Aa, Cry68Aa, Cry69Aa, Cry69Ab, Cry70Aa, Cry70Ba, Cry70Bb, Cry71Aa, Cry72Aa, Cry73Aa, or any combination of the foregoing. In some embodiments, the resistant plant pest has developed resistance to the pesticidal activity of the Cry1Ab protein in the Bt11 event (see U.S. Pat. No. 6,114,608), the Cry3A055 protein in the MIR604 event (see U.S. Pat. No. 8,884,102), the eCry3.1Ab protein in the 5307 event (see U.S. Pat. No. 10,428,393) and/or the mCry3A protein in the MZI098 event (see US Patent Application No. US20200190533). In some embodiments, the resistant plant pest has developed resistance to the pesticidal activity of the Bt11 event (see U.S. Pat. No. 6,114,608), the MIR604 event (see U.S. Pat. No. 8,884,102), the 5307 event (see U.S. Pat. No. 10,428,393) and/or the MZI098 event (see US Patent Application No. US20200190533).

In further embodiments, the resistant plant pest has developed resistance to the pesticidal activity of one or more Vip3 vegetative pesticidal proteins. Some structural features that identify a protein as being in the Vip3 class of proteins includes: 1) a size of about 80-88 kDa that is proteolytically processed by insects or trypsin to about a 62-66 kDa toxic core (Lee et al. 2003. Appl. Environ. Microbiol. 69:4648-4657); and 2) a highly conserved N-terminal secretion signal which is not naturally processed during secretion in B. thuringiensis. Non-limiting examples of members of the Vip3 class and their respective GenBank accession numbers, U.S. Patent or patent publication number are Vip3Aa1 (AAC37036), Vip3Aa2 (AAC37037), Vip3Aa3 (U.S. Pat. No. 6,137,033), Vip3Aa4 (AAR81079), Vip3Aa5 (AAR81080), Vip3Aa6 (AAR81081), Vip3Aa7 (AAK95326), Vip3 Aa8 (AAK97481), Vip3Aa9 (CAA76665), Vip3 Aa10 (AAN60738), Vip3Aa11 (AAR36859), Vip3Aa12 (AAM22456), Vip3Aa13 (AAL69542), Vip3Aa14 (AAQ12340), Vip3Aa15 (AAP51131), Vip3Aa16 (AAW65132), Vip3Aa17 (U.S. Pat. No. 6,603,063), Vip3Aa18 (AAX49395), Vip3Aa19 (DQ241674), Vip3 Aa19 (DQ539887), Vip3 Aa20 (DQ539888), Vip3Aa21 (ABD84410), Vip3Aa22 (AAY41427), Vip3Aa23 (AAY41428), Vip3Aa24 (BI 880913), Vip3Aa25 (EF608501), Vip3Aa26 (EU294496), Vip3Aa27 (EU332167), Vip3Aa28 (FJ494817), Vip3Aa29 (FJ626674), Vip3Aa30 (FJ626675), Vip3Aa31 (FJ626676), Vip3Aa32 (FJ626677), Vip3 Aa33 (GU073128), Vip3Aa34 (GU073129), Vip3 Aa35 (GU733921), Vip3Aa36 (GU951510), Vip3Aa37 (HM132041), Vip3Aa38 (HM117632), Vip3Aa39 (HM117631), Vip3Aa40 (HM132042), Vip3Aa41 (HM132043), Vip3Aa42 (HQ587048), Vip3 Aa43 (HQ594534), Vip3Aa44 (HQ650163), Vip3Ab1 (AAR40284), Vip3Ab2 (AAY88247), Vip3Acl (U.S. Patent Application Publication 20040128716), Vip3Ad1 (U.S. Patent Application Publication 20040128716), Vip3Ad2 (CAI43276), Vip3Ael (CAI43277), Vip3Af1 (U.S. Pat. No. 7,378,493), Vip3 Af2 (ADN08753), Vip3 Af3 (HM117634), Vip3Ag1 (ADN08758), Vip3Ag2 (FJ556803), Vip3Ag3 (HM117633), Vip3 Ag4 (HQ414237), Vip3Ag5 (HQ542193), Vip3Ah1 (DQ832323), Vip3Ba1 (AAV70653), Vip3Ba2 (HM117635), Vip3Bb1 (U.S. Pat. No. 7,378,493), Vip3Bb2 (AB030520) and Vip3Bb3 (ADI48120). In embodiments, the Vip3 protein is Vip3Aa (U.S. Pat. No. 6,137,033), for example, as represented by corn event MIR162 (U.S. Pat. Nos. 8,232,456; 8,455,720; and 8,618,272). In some embodiments, the resistant plant pest has developed resistance to the pesticidal activity of the event MIR162 (U.S. Pat. Nos. 8,232,456; 8,455,720; and 8,618,272).

In some embodiments, the resistant plant pest has developed resistance to the pesticidal activity of any one or more of the pesticidal proteins or dsRNAs present in any of the following events: the Bt11 event (see U.S. Pat. No. 6,114,608), the MIR604 event (see U.S. Pat. No. 8,884,102), the MIR162 event (see U.S. Pat. No. 8,232,456), the 5307 event (see U.S. Pat. No. 10,428,393), the MZIR098 event (see US Patent Application No. US20200190533), the TC1507 event (see U.S. Pat. No. 7,288,643), the DAS-59122-7 event (see U.S. Pat. No. 7,323,556), the MON810 event (see U.S. Pat. No. 6,713,259), the MON863 event (see U.S. Pat. No. 7,705,216), the MON89034 event (see U.S. Pat. No. 8,062,840), the MON88017 event (see U.S. Pat. No. 9,556,492), the DP-4114 event (see U.S. Pat. No. 9,725,772), the MON87411 event (see U.S. Pat. No. 9,441,240), the DP-032218-9 event (see US Patent Application No. US2015361447), the DP-033121-3 event (see US Patent Application No. US2015361446), the DP-023211-2 event (see PCT Publication No. WO2019209700), the MON95379 event (see US Patent Application No. US2020032289), the DBN9936 event (see PCT Publication No. WO2016173361), the DBN9501 event (see PCT Publication No. WO20207125), the GH5112E-117C event (see PCT Publication No. WO17/088480), LP007-1 (see Chinese Patent Application No. CN112852801), LP007-2 (Chinese Patent Application No. CN112831584), LP007-3 (Chinese Patent Application No. CN112877454), LP007-4 (Chinese Patent Application No. CN112831585), LP007-5 (Chinese Patent Application No. CN113151534), LP007-6 (Chinese Patent Application No. CN113151533), LP007-7 (Chinese Patent Application No. CN112852991), LP007-8 (CN113980958), Ruifeng8, ND207, or the Ruifeng125 event (see Chinese Patent Application No. CN105017391).

In embodiments, the resistant plant pest has developed resistance to the pesticidal activity of a toxin that is derived from sources other than B. thuringiensis. For example, the resistant plant pest has developed resistance to the pesticidal activity of an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus pesticidal protein, a Xenorhabdus spp. (such as X. nematophila or X. bovienii) pesticidal protein, a Photorhabdus spp. (such as P. luminescens or P. asymobiotica) pesticidal protein, a Brevibacillus spp. (such as B. laterosporous) pesticidal protein, a Lysinibacillus spp. (such as L. sphearicus) pesticidal protein, a Chromobacterium spp. (such as C. subtsugae or C. piscinae) pesticidal protein, a Yersinia spp. (such as Y. entomophaga) pesticidal protein, a Paenibacillus spp. (such as P. propylaea) pesticidal protein, a Clostridium spp. (such as C. bifermentans) pesticidal protein, a Pseudomonas spp. (such as P. fluorescens) and a lignin. In other embodiments, the resistant plant pest has developed resistance to at least one pesticidal protein derived from a pesticidal toxin complex (Tc) from Photorhabdus, Xenorhabus, Serratia, or Yersinia. In other embodiments, the resistant plant pest has developed resistance to an ADP-ribosyltransferase derived from a pesticidal bacteria, such as Photorhabdus ssp. In other embodiments, the resistant plant pest has developed resistance to a VIP protein, such as VIP1 and/or VIP2 from B. cereus. In still other embodiments, the resistant plant pest has developed resistance to a binary toxin derived from a pesticidal bacteria, such as ISP1A and ISP2A from B. laterosporous or BinA and BinB from L. sphaericus. In still other embodiments, the resistant plant pest has developed resistance to a protein that has been engineered or may be a hybrid or chimera of any of the preceding pesticidal proteins.

Other embodiments encompass a resistant plant pest that has developed resistance to one or more other pest controls agents selected from DIG-657 (US Patent Publication 2015366211); PtIP-96 (US Patent Publication 2017233440); PIP-72 (US Patent Publication US2016366891); PIP-83 (US Patent Publication 2016347799); PIP-50 (US Patent Publication 2017166921); IPD73 (US Patent Publication 2019119334); IPD090 (US Patent Publication 2019136258); IPD80 (US Patent Publication 2019256563); IPD078, IPD084, IPD086, IPD087, IPD089 (US Patent Publication 2020055906); IPD093 (International Application Publication WO2018111551); IPD059 (International Application Publication WO2018232072); IPD113 (International Application Publication WO2019178042); IPD121 (International Application Publication WO2018208882); IPD110 (International Application Publication WO2019178038); IPD103 (International Application Publication WO2019125717); IPD092; IPD095; IPD097; IPD099; IPD100, IPD105; IPD106; IPD107; IPD111; IPD112 (International Application Publication WO2020055885); IPD102 (International Application Publication WO2020076958) Cry1B.868 and Cry1Da_7 (US Patent Publication 2020-032289); TIC107 (U.S. Pat. No. 8,049,071); Cry2Ab and Cry1A.105 (U.S. Pat. No. 10,584,391); Cry1F, Cry34Ab1, Cry35Ab1 (U.S. Pat. No. 10,407,688); TIC6757, TIC7472, TIC7473, TIC6757 (US Patent Publication 2017058294); TIC3668, TIC3669, TIC3670, TIC4076, TIC4078, TIC4260, TIC4346, TIC4826, TIC4861, TIC4862, TIC4863, TIC-3668 (US Patent Publication 2016319302); TIC7040, TIC7042, TIC7381, TIC7382, TIC7383, TIC7386, TIC7388, TIC7389 (US Patent Publication 2018291395); TIC7941 (US Patent Publication 2020229445) TIC836, TIC860, TIC867, TIC868, TIC869, and TIC1100 (International Application Publication WO2016061391), TIC2160 (International Application Publication WO2016061392), ET66, TIC400, TIC800, TIC834, TIC1415, AXMI-001, AXMI-002, AXMI-030, AXMI-035, AND AXMI-045 (US Patent Publication 20130117884), AXMI-52, AXMI-58, AXMI-88, AXMI-97, AXMI-102, AXMI-112, AXMI-117, AXMI-100 (US Patent Publication 201-0310543), AXMI-115, AXMI-113, AXMI-005 (US Patent Publication 20130104259), AXMI-134 (US Patent Publication 20130167264), AXMI-150 (US Patent Publication 20100160231), AXMI-184 (US Patent Publication 20100004176), AXMI-196, AXMI-204, AXMI-207, AXMI-209 (US Patent Publication 2011-0030096), AXMI-218, AXMI-220 (US Patent Publication 20140245491), AXMI-221z, AXMI-222z, AXMI-223z, AXMI-224z, AXMI-225z (US Patent Publication 20140196175), AXMI-238 (US Patent Publication 20140033363), AXMI-270 (US Patent Publication 20140223598), AXMI-345 (US Patent Publication 20140373195), AXMI-335 (International Application Publication WO2013134523), DIG-3 (US Patent Publication 20130219570), DIG-5 (US Patent Publication 20100317569), DIG-11 (US Patent Publication 20100319093), AfIP-1A (US Patent Publication 20140033361), AfIP-1B (US Patent Publication 20140033361), PIP-1APIP-1B (US Patent Publication 20140007292), PSEEN3174 (US Patent Publication 20140007292), AECFG-592740 (US Patent Publication 20140007292), Pput_1063 (US Patent Publication 20140007292), DIG-657 (International Application Publication WO2015195594), Pput_1064 (US Patent Publication 20140007292), GS-135 (US Patent Publication 20120233726), GS153 (US Patent Publication 20120192310), GS154 (US Patent Publication 20120192310), GS155 (US Patent Publication 20120192310), DIG-911 and DIG-180 (US Patent Publication No. 20150264940); and the like.

In some embodiments, the resistant plant pest has developed resistance to an agent that is non-proteinaceous, for example, an interfering RNA molecule such as a dsRNA, which can be expressed transgenically or applied as part of a composition (e.g., using topical methods). An interfering RNA typically comprises at least a first RNA fragment against a target gene, a spacer sequence, and a second RNA fragment which is complementary to the first, so that a double-stranded RNA structure can be formed. RNA interference (RNAi) occurs when an organism recognizes double-stranded RNA (dsRNA) molecules and hydrolyzes them. The resulting hydrolysis products are small RNA fragments of about 19-24 nucleotides in length, called small interfering RNAs (siRNAs). The siRNAs then diffuse or are carried throughout the organism, including across cellular membranes, where they hybridize to mRNAs (or other RNAs) and cause hydrolysis of the RNA. Interfering RNAs are recognized by the RNA interference silencing complex (RISC) into which an effector strand (or “guide strand”) of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes from which the mRNAs were transcribed. Interfering RNAs are known in the art to be useful for insect control (see, for example, publication WO2013/192256, incorporated by reference herein). An interfering RNA designed for use in insect control produces a non-naturally occurring double-stranded RNA, which takes advantage of the native RNAi pathways in the insect to trigger down-regulation of target genes that may lead to the cessation of feeding and/or growth and may result in the death of the insect pest. The interfering RNA molecule may confer insect resistance against the same target pest as the disclosed proteins or may target a different pest. The targeted insect plant pest may feed by chewing, sucking, or piercing. Interfering RNAs are known in the art to be useful for insect control. In embodiments, the dsRNA against which the resistant pest has developed resistance is described in US Patent Publications 20190185526, 2018020028 or 20190177736. In embodiments, the dsRNA is described in U.S. Pat. Nos. 9,238,8223, 9,340, 797, or 8,946,510. In embodiments, the dsRNA is described in U.S. Patent Publications 20200172922, 20110054007, 20140275208, 20160230185, or 20160230186.

### Chemical Pesticides

In some embodiments, the population of plant pests that are resistant to a pest control agent include pests which have developed resistance to one or more chemical pesticides, which is optionally a seed coating. Non-limiting examples of chemical pesticides include pyrethroids, carbamates, neonicotinoids, neuronal sodium channel blockers, pesticidal macrocyclic lactones, gamma-aminobutyric acid (GABA) antagonists, pesticidal ureas and juvenile hormone mimics. In other embodiments, the chemical pesticide is one or more of abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad. In still other embodiments, the chemical pesticide is selected from one or more of cypermethrin, cyhalothrin, cyfluthrin and beta-cyfluthrin, esfenvalerate, fenvalerate, tralomethrin, fenothicarb, methomyl, oxamyl, thiodicarb, clothianidin, imidacloprid, thiacloprid, indoxacarb, spinosad, abamectin, avermectin, emamectin, endosulfan, ethiprole, fipronil, flufenoxuron, triflumuron, diofenolan, pyriproxyfen, pymetrozine and amitraz.

### Plant Pests

The disclosed methods are useful for identifying a gene, trait, or genetic locus associated resistance of a plant pest to a pesticidal agent. In some embodiments, the plant pest that has developed resistance is a Lepidopteran, Coleopteran, Hemipteran, Dipteran, Lygus spp., and/or other piercing and sucking insects, for example of the order Orthoptera or Thysanoptera.

Non-limiting examples of a Coleopteran pest include: Diabrotica spp, such as D. barberi (northern corn rootworm), D. virgifera virgifera (western corn rootworm), D. undecimpunctata howardii (southern corn rootworm), D. balteata (banded cucumber beetle), D. undecimpunctata undecimpunctata (western spotted cucumber beetle), D. significata (3-spotted leaf beetle), D. speciosa (cucurbit beetle), D. virgifera zeae (Mexican corn rootworm), D. beniensis, D. cristata, D. curviplustalata, D. dissimilis, D. elegantula, D. emorsitans, D. graminea, D. hispanloe, D. lemniscata, D. linsleyi, D. milleri, D. nummularis, D. occlusal, D. porrecea, D. scutellata, D. tibialis, D. trifasciata and/or D. viridula, Leptinotarsa spp, such as L. decemlineata (Colorado potato beetle), Chrysomela spp, such as C. scripta (cottonwood leaf beetle), Hypothenemus spp. such as H. hampei (coffee berry borer), Sitophilus spp, such as S. zeamais (maize weevil), Epitrix spp, such as E. hirtipennis (tobacco flea beetle) and/or E. cucumeris (potato flea beetle), Phyllotreta spp, such as P. cruciferae (crucifer flea beetle) and/or P. pusilla (western black flea beetle), Anthonomus spp, such as A. eugenii (pepper weevil), Hemicrepidus spp, such as H. memnonius (wireworms), Melanotus spp, such as M. communis (wireworm), Ceutorhychus spp. such as C. assimilis (cabbage seedpod weevil), Phyllotreta spp, such as P. cruciferae (crucifer flea beetle), Aeolus spp, such as A. mellillus (wireworm), Aeolus spp, such as A. mancus (wheat wireworm), Horistonotus spp, such as H. uhlerii (sand wireworm), Sphenophorus spp, such as S. maidis (maize billbug), S. zeae (timothy billbug), S. parvulus (bluegrass billbug), and S. callosus (southern corn billbug), Phyllophaga spp. (White grubs), Chaetocnema spp, such as C. pulicaria (corn flea beetle), Popillia spp, such as P. japonica (Japanese beetle), Epilachna spp, such as E. varivestis (Mexican bean beetle), Cerotoma spp, such as C. trifurcate (Bean leaf beetle), Epicauta spp, such as E. pestifera and E. lemniscata (Blister beetles), Holotrichia spp, such as H. diomphalia Bates (Northeast larger black chafer), or any combination of the foregoing. In some embodiments, the plant pest is one or more of the following Hemiptera pests: Chinavia hilaris (green stink bug), Anasa tristis De Geer (squash bug), Blissus leucopterus (chinch bug), Corythuca gossypii Fabricius (cotton lace bug), Cyrtopeltis modesta Distant (tomato bug), Dysdercus suturellus Hernch-Schaffer (cotton stainer), Euschistus servus Say (brown stink bug), E. variolarius Palisot de Beauvois (one-spotted stink bug), Graptostethus spp. (complex of seed bugs), Leptoglossus corculus Say (leaf-footed pine seed bug), Lygus lineolaris Palisot de Beauvois (tarnished plant bug), L. Hesperus Knight (Western tarnished plant bug), L. pratensis Linnaeus (common meadow bug), L. rugulipennis Poppius (European tarnished plant bug), Lygocoris pabulinus Linnaeus (common green capsid), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper), Calocoris norvegicus Gmelin (strawberry bug), Orthops campestris Linnaeus, Plesiocoris rugicollis Fallen (apple capsid), Cyrtopeltis modestus Distant (tomato bug), Cyrtopeltis notatus Distant (suckfly), Spanagonicus albofasciatus Reuter (whitemarked fleahopper), Diaphnocoris chlorionis Say (honeylocust plant bug), Labopidicola allii Knight (onion plant bug), Pseudatomoscelis seriatus Reuter (cotton fleahopper), Adelphocoris rapidus Say (rapid plant bug), Poecilocapsus lineatus Fabricius (four-lined plant bug), Nysius ericae Schilling (false chinch bug), Nysius raphanus Howard (false chinch bug), Nezara viridula Linnaeus (Southern green stink bug), Eurygaster spp., Coreidae spp., Pyrrhocoridae spp., Tinidae spp., Blostomatidae spp., Reduviidae spp. and Cimicidae spp., or any combination of the foregoing. In some embodiments, the plant pest is one or more of the following Diptera pests: Liriomyza spp, such as L. trifolii (leafminer) and L. sativae (vegetable leafminer), Scrobipalpula spp, such as S. absoluta (tomato leafminer), Delia spp, such as D. platura (seedcorn maggot), D. brassicae (cabbage maggot) and D. radicum (cabbage root fly), Psilia spp, such as P. rosae (carrot rust fly), and Tetanops spp, such as T. myopaeformis (sugarbeet root maggot).

Non-limiting examples of Orthoptera pests include: Melanoplus spp, such as M. differentialis (Differential grasshopper), M. femurrubrum (Redlegged grasshopper), M. bivittatus (Twostriped grasshopper), or any combination of the foregoing. Non-limiting examples of Thysanoptera pests include: Frankliniella spp, such as F. occidentalis (western flower thrips) and F. fusca (tobacco thrips), and Thrips spp, such as T. tabaci (onion thrips), or T. palmi (melon thrips).

Non-limiting examples of a Lepidopteran pest include: Spodoptera spp, such as S. frugiperda (fall armyworm), S. littoralis (Egyptian cotton leafworm), S. ornithogalli (yellowstriped armyworm), S. praefica (western yellowstriped armyworm), S. eridania (southern armyworm), S. litura (Common cutworm/Oriental leafworm), S. cosmioides (black armyworm), S. exempta (African armyworm), S. mauritia (lawn armyworm) and/or S. exigua (beet armyworm), Ostrinia spp, such as O. nubilalis (European corn borer) and/or O. furnacalis (Asian corn borer), Plutella spp, such as P. xylostella (diamondback moth), Agrotis spp, such as A. ipsilon (black cutworm), A. segetum (common cutworm), A. gladiaria (claybacked cutworm), and/or A. orthogonia (pale western cutworm), Striacosta spp, such as S. albicosta (western bean cutworm), Helicoverpa spp, such as H. zea (corn earworm/soybean podworm), H. punctigera (native budworm), and/or H. armigera (cotton bollworm), Heliothis spp, such as H. virescens (tobacco budworm), Diatraea spp, such as D. grandiosella (southwestern corn borer) and/or D. saccharalis (sugarcane borer), Trichoplusia spp, such as T. ni (cabbage looper), Sesamia spp. such as S. nonagroides (Mediterranean corn borer), S. inferens (Pink stem borer) and/or S. calamistis (pink stem borer), Pectinophora spp, such as P. gossypiella (pink bollworm), Cochylis spp, such as C. hospes (banded sunflower moth), Manduca spp, such as M. sexta (tobacco hornworm) and/or M. quinquemaculata (tomato hornworm), Elasmopalpus spp, such as E. lignosellus (lesser cornstalk borer), Pseudoplusia spp, such as P. includens (soybean looper), Anticarsia spp, such as A. gemmatalis (velvetbean caterpillar), Plathypena spp, such as P. scabra (green cloverworm), Pieris spp, such as P. brassicae (cabbage butterfly), Papaipema spp. such as P. nebris (stalk borer), Pseudaletia spp, such as P. unipuncta (common armyworm), Peridroma spp, such as P. saucia (variegated cutworm), Keiferia spp, such as K. lycopersicella (tomato pinworm), Artogeia spp, such as A. rapae (imported cabbageworm), Phthorimaea spp. such as P. operculella (potato tuberworm), Chrysodeixis spp, such as C. includens (soybean looper), Feltia spp, such as F. ducens (dingy cutworm), Chilo spp, such as C. suppressalis (striped stem borer), C. Agamemnon (oriental corn borer), C. venosatus (spotted borer), and C. partellus (spotted stalk borer), Cnaphalocrocis spp, such as C. medinalis (rice leaffolder), Conogethes spp, such as C. punctiferalis (Yellow peach moth), Mythimna spp, such as M. separata (Oriental armyworm), Athetis spp, such as A. lepigone (Two-spotted armyworm), Busseola spp, such as B. fusca (maize stalk borer), Etiella spp, such as E. zinckenella (pulse pod borer), Leguminivora spp, such as L. glycinivorella (soybean pod borer), Matsumuraeses spp. such as M. phaseoli (adzuki pod worm), Omiodes spp, such as O. indicata (Soybean leaffolder/Bean-leaf webworm), Rachiplusia spp, such as R. nu (sunflower Looper), Maruca spp. such as M. Testulalis Geyer (Bean pod borer), Monolepta spp, such as M. hieroglyphica (Double-spotted leaf beetle),

Other resistant pests may include one or more of the following: Phyllophaga spp., Rhopalosiphum maidis, Pratylenchus penetrans, Melanotus cribulosus, Cyclocephala lurida, Limonius californicus, Tetranychus urticae, Haplothrips aculeatus, Tetranychus truncates, Anomala corpulenta, Oedaleus infernalis, Frankliniella tenuicornis, Tetranychus cinnabarinus, Aiolopus thalassinus tamulus, Trachea tokionis, Laodelphax striatellus, Holotrichia oblita, Dichelops furcatus, Diloboderus abderu, Dalbulus maidis, Astylus variegathus, Scaptocoris castanea, Locusta migratoria manilensis, Agriotes lineatus, Peregrinus maidis, Oscinella frit, Frankliniella williamsi, Zyginidia manaliensis, Atherigona soccata, Nicentrites testaceipes, Myllocerus undecimpustulatus, Atherigona naquii, Amsecta albistriga, Plodia interpuctella, Melanotus caudex, Microtermes spp., Atherigona oryzae, Tanymecus dilaticollis, Delphacodes kuschelli, Lepidiota stigma, Phyllophaga hellery, Tribolium castaneum, Pelopidas mathias, Oxya chinensis (Thunberg), Stenocranus pacificus, Scutigerella immaculata, Chrysodeixis chalcites, Euproctis sp. (Lymantriidae), Phyllotreata spp. (undulata), Reptalus panzer, Cyrtacanthacris tartarica Linnaeus, Orgyia postica, Dactylispa lameyi, Patanga succincta Johanson, Tetranychus spp., Calomycterus sp., Adoretus compressus Weber, and Paratetranychus stickney

Insects of the order Hemiptera include but are not limited to Chinavia hilaris (green stink bug); Anasa tristis De Geer (squash bug); Blissus leucopterus (chinch bug); Corythuca gossypii Fabricius (cotton lace bug); Cyrtopeltis modesta Distant (tomato bug); Dysdercus suturellus Hern ch-Schaffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper), Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp, and Cimicidae spp.

Insects in the order Diptera include but are not limited to Liriomyza spp, such as L. trifolii (leafminer) and L. sativae (vegetable leafminer); Scrobipalpula spp, such as S. absoluta (tomato leafminer); Delia spp, such as D. platura (seedcorn maggot), D. brassicae (cabbage maggot) and D. radicum (cabbage root fly); Psilia spp, such as P. rosae (carrot rust fly); Tetanops spp. such as T. myopaeformis (sugarbeet root maggot); and any combination of the foregoing.

Insects in the order Orthoptera include but are not limited to Melanoplus spp, such as M. differentialis (Differential grasshopper), M. femurrubrum (Redlegged grasshopper), M. bivittatus (Twostriped grasshopper); and any combination thereof. Insects in the order Thysanoptera include but are not limited to Frankliniella spp, such as F. occidentalis (western flower thrips) and F. fusca (tobacco thrips); and Thrips spp, such as T. tabaci (onion thrips), T. palmi (melon thrips); and any combination of the foregoing.

The present invention is also useful for identifying the gene, trait, or genetic locus associated with resistance of a nematode to a pesticidal agent, e.g. a nematicide. The term “nematode” as used herein encompasses any organism that is now known or later identified that is classified in the animal kingdom, phylum Nematoda, including without limitation nematodes within class Adenophorea (including for example, orders Enoplida, Isolaimida, Mononchida, Dorylaimida, Trichocephalida, Mermithida, Muspiceida, Araeolaimida, Chromadorida, Desmoscolecida, Desmodorida and Monhysterida) and/or class Secernentea (including, for example, orders Rhabdita, Strongylida, Ascaridida, Spirurida, Camallanida, Diplogasterida, Tylenchida and Aphelenchida). Nematodes include but are not limited to parasitic nematodes such as root-knot nematodes, cyst nematodes and/or lesion nematodes. Exemplary genera of nematodes according to the present disclosure include but are not limited to, Meloidogyne (root-knot nematodes), Heterodera (cyst nematodes), Globodera (cyst nematodes), Radopholus (burrowing nematodes), Rotylenchulus (reniform nematodes), Pratylenchus (lesion nematodes), Aphelenchoides (foliar nematodes), Helicotylenchus (spiral nematodes), Hoplolaimus (lance nematodes), Paratrichodorus (stubby-root nematodes), Longidorus, Nacobbus (false root-knot nematodes), Subanguina, Belonlaimus (sting nematodes), Criconemella, Criconemoides (ring nematodes), Ditylenchus, Dolichodorus, Hemicriconemoides, Hemicycliophora, Hirschmaniella, Hypsoperine, Macroposthonia, Melinius, Punctodera, Quinisulcius, Scutellonema, Xiphinema (dagger nematodes), Tylenchorhynchus (stunt nematodes), and Tylenchulus, Bursaphelenchus (round worms).

Exemplary plant parasitic nematodes according to the present disclosure include, but are not limited to, Belonolaimus gracilis, Belonolaimus longicaudatus, Bursaphelenchus xylophilus (pine wood nematode), Criconemoides ornata, Ditylenchus destructor (potato rot nematode), Ditylenchus dipsaci (stem and bulb nematode), Globodera pallida (potato cyst nematode), Globodera rostochiensis (golden nematode), Heterodera glycines (soybean cyst nematode), Heterodera schachtii (sugar beet cyst nematode); Heterodera zeae (corn cyst nematode), Heterodera avenae (cereal cyst nematode), Heterodera carotae, Heterodera trifolii, Hoplolaimus columbus, Hoplolaimus galeatus, Hoplolaimus magnistylus, Longidorus breviannulatus, Meloidogyne arenaria, Meloidogyne chitwoodi, Meloidogyne hapla, Meloidogyne incognita, Meloidogyne javanica, Mesocriconema xenoplax, Nacobbus aberrans, Naccobus dorsalis, Paratrichodorus christiei, Paratrichodorus minor, Pratylenchus brachyurus, Pratylenchus crenatus, Pratylenchus hexincisus, Pratylenchus neglectus, Pratylenchus penetrans, Pratylenchus projectus, Pratylenchus scribneri, Pratylenchus tenuicaudatus, Pratylenchus thornei, Pratylenchus zeae, Punctodera chaccoensis, Quinisulcius acutus, Radopholus similis, Rotylenchulus reniformis, Tylenchorhynchus dubius, Tylenchulus semipenetrans (citrus nematode), Siphinema americanum, X. Mediterraneum, and any combination of the foregoing.

### EXAMPLES

Embodiments of the invention can be better understood by reference to the following detailed examples. The foregoing and following description of embodiments of the invention and the various embodiments are not intended to limit the claims but are rather illustrative thereof. Therefore, it will be understood that the claims are not limited to the specific details of these examples. It will be appreciated by those skilled in the art that other embodiments of the invention may be practiced without departing from the spirit and the scope of the disclosure, the scope of which is defined by the appended claims. Art recognized recombinant DNA and molecular cloning techniques may be found in, for example, J. Sambrook, et al., Molecular Cloning: A Laboratory Manual, 4th Ed., Cold Spring Harbor, NY: Cold Spring Harbor Laboratory Press (2012); by T. J. Silhavy, M. L. Berman, and L. W. Enquist, Experiments with Gene Fusions, Cold Spring Harbor Laboratory, Cold Spring Harbor, NY (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### EXPERIMENTAL EXAMPLES

### Background

In Lepidopteran larvae, the midgut epithelium is a complex of digestive, secretory, and regenerative cells. It consists of a single layer of columnar and goblet cells with stem cells lying along the basal sides (Loeb 2010). The larval midgut is responsible for food digestion, nutrients absorption and ion balance regulation. The midgut is also the target site of many insecticidal proteins, such as Bt toxins isolated from Bacillus thuringiensis (Federici 1993). Cry toxins, the major class of insect control traits on the market, bind to the brush-border membrane of columnar cells to attain their insecticidal activities by lysing cells (Nagamatsu et al. 1998). To understand resistance mechanism of insecticidal proteins and identify their receptors, a single cell sequencing method was developed to identify receptors of insect control traits from midgut columnar cells.

### Example 1. Isolation of Primary Midgut Cells from Fall Armyworm (FAW)

Recent studies show that ATP-binding cassette (ABC) transporters serve as the major receptor for several insect control traits based on linkage mapping of resistant strains collected from field or by cell-based validation. Columnar cells (enterocytes) in insect midgut secrete digestive enzymes, absorb and transport nutrients. Multiple line of evidence suggests that receptors of Cry toxin families are localized on the microvilli of columnar cells. FAW midguts were dissociated to single cells in a method adapted from Hakim et al. (2009) for single cell sequencing.

Briefly, midgut tissues were dissected from surface sterilized 5^{th} instar FAW larvae in LPS buffer (NaCl 178 mM, KCl 4.3 mM, CaCl₂) 4.3 mM, NaHCO₃3.8 mM, 0.5% gentamicin, 0.01% antibiotic-antimycotic solution, pH 6.5). Fifteen midguts were collected in a 35-mm petri dish containing LPS. Midgut tissues were transferred and rinsed in a new petri dish with LPS. The process was repeated for three times to remove food and tissue debris.

To release columnar and other epithelial cells, midgut tissue was placed in a Falcon 70-μm cell strainer (Corning, NY) sitting in a 50-mm petri dish filled with LPS. The midgut tissue was stirred on an orbital shaker for 1 hour. The freed cells were then transferred to a conical bottom tube and centrifuged at 200 g for 10 min. Cell pellet was washed twice by LPS before resuspended in Hank's balanced salt solution (HBSS) for sequencing. Isolated primary cells were mixed with 0.4% Trypan Blue (Thermo Fisher) to assess cell viability and density with a hemocytometer. Microscopic analysis confirmed the presence of columnar cells with microvilli (among other epithelial cells in the cell suspension).

### Example 2. Library Preparation and Single Cell Sequencing

The isolated columnar cells were processed through 10× Chromium single cell platform following the protocol of 10× Genomics (Pleasanton, CA). Briefly, Chromium Next GEM Single Cell 3′ LT Reagent Kit v3.1 (Dual Index) was used for gene expression profiling based on 3′-RNASeq. Two thousand columnar cells were loaded on a single-cell chip. Then, the chip was put into a 10× Chromium controller for cell partitioning and droplet generation. Cells were encapsulated into single cell GEMs (Gel Beads in Emulsion). Reverse transcription was followed within the droplet where all generated cDNAs share a common 10× Barcode. Subsequently, cDNA was recovered by demulsification and bead purification. Amplified cDNA was used for Illumina library construction with P5 and P7 primers containing i5 and i7 index sequences. The libraries were quantified by an Agilent Tape Station 4200.

Single cell libraries were sequenced by an Illumina MiSeq system with the following number of cycles: R1 (28 cycles), i7 (10 cycles), i5 (10 cycles) and R2 (90 cycles). Paired-end reads produced Read 1 (R1) containing the 16 bp 10× Barcode and 12 bp Unique Molecular Identifiers (UMI) and Read 2 (R2) including 90 bp sequence from the 3′ end of cDNA. The present invention can be adapted to accommodate sequence reads longer than 90 bp.

### Example 3. Identifying the Marker Genes of Columnar Cells

Fifteen Drosophila marker genes (Hung et al. 2020) of anterior enterocyte, differentiated enterocyte, middle enterocyte or enterocyte-like cells were scanned against a proprietary transcriptome assembled from Brazilian FAW sequences. FAW orthologs of eleven Drosophila marker genes were found. Top hits with the highest nucleotide identity of FAW orthologs to 11 Drosophila marker genes are listed in Table 1. Nucleotide identity between FAW and Drosophila of these marker genes is between 12% and 62%. The discovered FAW marker genes were used to cluster columnar cells from the rest of the epithelial cell population based on FAW single cell sequencing results.

Additional marker genes of midgut stem and goblet cells can be identified by the similar approach.

TABLE 1
FAW orthologs of the Drosophila enterocyte marker genes.

| | | | | Nucleotide |
|---|---|---|---|---|
| SEQ | | Drosophila | | Identity (%) |
| ID | Drosophila | Transcript | | to Drosophila |
| NO: | Markers | ID | BR FAW Orthologs Transcript ID | transcript |
| 1 | Oatp58Dc | FBtr0071777 | S_FRUGIPERDA_BR_REF_2_TRANSCRIPT\|Sf.BR.2.1.0001358.t2 | 15 |
| 2 | Oatp58Dc | FBtr0071777 | S_FRUGIPERDA_BR_REF_2_TRANSCRIPT\|Sf.BR.2.1.0001433.t4 | 30 |
| 3 | Oatp58Dc | FBtr0071777 | S_FRUGIPERDA_BR_REF_2_TRANSCRIPT\|Sf.BR.2.1.0015985.t4 | 25 |
| 4 | Alp2 | FBtr0071782 | S_FRUGIPERDA_BR_REF_2_TRANSCRIPT\|Sf.BR.2.1.0000303.t11 | 45 |
| 5 | Alp2 | FBtr0071782 | S_FRUGIPERDA_BR_REF_2_TRANSCRIPT\|Sf.BR.2.1.0000305.t2 | 46 |
| 6 | Alp2 | FBtr0071782 | S_FRUGIPERDA_BR_REF_2_TRANSCRIPT\|Sf.BR.2.1.0000898.t6 | 32 |
| 7 | lab | FBtr0081696 | S_FRUGIPERDA_BR_REF_2_TRANSCRIPT\|Sf.BR.2.1.0013982.t3 | 32 |
| 8 | lab | FBtr0081696 | S_FRUGIPERDA_BR_REF_2_TRANSCRIPT\|Sf.BR.2.1.0014280.t3 | 24 |
| 9 | lab | FBtr0081696 | S_FRUGIPERDA_BR_REF_2_TRANSCRIPT\|Sf.BR.2.1.0014283.t6 | 31 |
| 10 | Vha100-4 | FBtr0083658 | S_FRUGIPERDA_BR_REF_2_TRANSCRIPT\|Sf.BR.2.1.0008778.t3 | 62 |
| 11 | Vha100-4 | FBtr0083658 | S_FRUGIPERDA_BR_REF_2_TRANSCRIPT\|Sf.BR.2.1.0008926.t3 | 47 |
| 12 | Jon99Ciii | FBtr0085502 | S_FRUGIPERDA_BR_REF_2_TRANSCRIPT\|Sf.BR.2.1.0009340.t12 | 41 |
| 13 | Jon99Ciii | FBtr0085502 | S_FRUGIPERDA_BR_REF_2_TRANSCRIPT\|Sf.BR.2.1.0009341.t2 | 28 |
| 14 | Jon99Ciii | FBtr0085502 | S_FRUGIPERDA_BR_REF_2_TRANSCRIPT\|Sf.BR.2.1.0009342.t7 | 41 |
| 15 | Alp4 | FBtr0085733 | S_FRUGIPERDA_BR_REF_2_TRANSCRIPT\|Sf.BR.2.1.0000303.t13 | 31 |
| 16 | Alp4 | FBtr0085733 | S_FRUGIPERDA_BR_REF_2_TRANSCRIPT\|Sf.BR.2.1.0008808.t54 | 18 |
| 17 | Alp4 | FBtr0085733 | S_FRUGIPERDA_BR_REF_2_TRANSCRIPT\|Sf.BR.2.1.0009050.t8 | 34 |
| 18 | Smvt | FBtr0085860 | S_FRUGIPERDA_BR_REF_2_TRANSCRIPT\|Sf.BR.2.1.0004552.t8 | 12 |
| 19 | Smvt | FBtr0085860 | S_FRUGIPERDA_BR_REF_2_TRANSCRIPT\|Sf.BR.2.1.0007218.t6 | 21 |
| 20 | Amy-p | FBtr0087004 | S_FRUGIPERDA_BR_REF_2_TRANSCRIPT\|Sf.BR.2.1.0006457.t8 | 59 |
| 21 | Amy-p | FBtr0087004 | S_FRUGIPERDA_BR_REF_2_TRANSCRIPT\|Sf.BR.2.1.0010586.t1 | 23 |
| 22 | Amy-p | FBtr0087004 | S_FRUGIPERDA_BR_REF_2_TRANSCRIPT\|Sf.BR.2.1.0012676.t1 | 56 |
| 23 | lambdaTry | FBtr0088121 | S_FRUGIPERDA_BR_REF_2_TRANSCRIPT\|Sf.BR.2.1.0003735.t3 | 38 |
| 24 | lambdaTry | FBtr0088121 | S_FRUGIPERDA_BR_REF_2_TRANSCRIPT\|Sf.BR.2.1.0005898.t17 | 33 |
| 25 | lambdaTry | FBtr0088121 | S_FRUGIPERDA_BR_REF_2_TRANSCRIPT\|Sf.BR.2.1.0009685.t1 | 43 |
| 26 | betaTry A | FBtr0088122 | S_FRUGIPERDA_BR_REF_2_TRANSCRIPT\|Sf.BR.2.1.0000923.t2 | 46 |
| 27 | betaTry A | FBtr0088122 | S_FRUGIPERDA_BR_REF_2_TRANSCRIPT\|Sf.BR.2.1.0005900.t2 | 40 |
| 28 | betaTry B | FBtr0345928 | S_FRUGIPERDA_BR_REF_2_TRANSCRIPT\|Sf.BR.2.1.0005898.t16 | 44 |

### Example 4. Determining Marker Gene Expression in Columnar Cells of FAW by Single Cell Sequencing

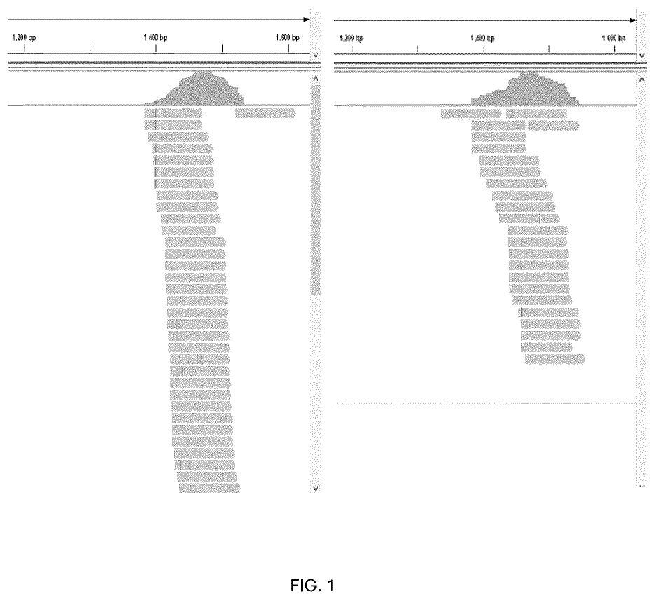

A FASTQ file with R2 reads from midgut cells of the resistant BR (Brazilian) or susceptible NA (North American) FAW midgut transcripts was mapped to the 11 marker genes identified in the Example 3. Read alignment was performed using the HISAT2 (Kim et al. 2019) with default parameters. The reads aligned to the individual marker gene were computed by the HISAT2 as shown in the Table 2. Samtools (Danecek et al. 2021) was used for file conversion and clean up to obtain final mapped reads of marker genes. Finally, the overlapping transcript reads were visualized using Integrative Genomics Viewer (IGV) (Robinson et al. 2011). R2 reads of 11 columnar cell markers in BR and NA FAW midgut fall into a wide range (1-1418). Among them, two marker genes (SEQ ID NO:3 and 19) only have less than 10 mapped reads, which suggests their extremely low expression level and, thus, were excluded from further cluster analysis. The remaining 9 marker genes demonstrated relative abundance between BR and NA FAW. FIG. 1 shows R2 reads from single cell sequencing of NA FAW (left panel) and BR FAW (right panel) midgut mapped to the columnar cell marker Sf.BR.2.1.0000305.t2 (SEQ ID NO: 5).

TABLE 2
Expression (R2 reads) of FAW columnar cell
marker genes in BR FAW and NA FAW midgut.

| | | Reads | Reads |
|---|---|---|---|
| Seq | | of BR | of NA |
| ID | Transcript ID of FAW Columnar Cell Markers | FAW | FAW |
| 3 | S_FRUGIPERDA_BR_REF_2_TRANSCRIPT\|Sf.BR.2.1.0015985.t4 | 3 | 6 |
| 5 | S_FRUGIPERDA_BR_REF_2_TRANSCRIPT\|Sf.BR.2.1.0000305.t2 | 26 | 64 |
| 7 | S_FRUGIPERDA_BR_REF_2_TRANSCRIPT\|Sf.BR.2.1.0013982.t3 | 22 | 18 |
| 10 | S_FRUGIPERDA_BR_REF_2_TRANSCRIPT\|Sf.BR.2.1.0008778.t3 | 47 | 82 |
| 14 | S_FRUGIPERDA_BR_REF_2_TRANSCRIPT\|Sf.BR.2.1.0009342.t7 | 1193 | 1418 |
| 15 | S_FRUGIPERDA_BR_REF_2_TRANSCRIPT\|Sf.BR.2.1.0000303.t13 | 42 | 93 |
| 19 | S_FRUGIPERDA_BR_REF_2_TRANSCRIPT\|Sf.BR.2.1.0007218.t6 | 1 | 1 |
| 20 | S_FRUGIPERDA_BR_REF_2_TRANSCRIPT\|Sf.BR.2.1.0006457.18 | 76 | 159 |
| 23 | S_FRUGIPERDA_BR_REF_2_TRANSCRIPT\|Sf.BR.2.1.0003735.t3 | 120 | 106 |
| 26 | S_FRUGIPERDA_BR_REF_2_TRANSCRIPT\|Sf.BR.2.1.0000923.t2 | 690 | 729 |
| 28 | S_FRUGIPERDA_BR_REF_2_TRANSCRIPT\|Sf.BR.2.1.0005898.t16 | 818 | 664 |

### Example 5. Cluster Analysis of the Midgut Cell Population

Cell type specific clusters are defined by annotating marker genes, as outlined in the Example 3 and 4, of midgut epithelial cells. After the midgut single cell sequencing data is quality checked and poor-quality cells are removed, the raw reads from sequencing run are processed into distinct cell clusters by bioinformatics tools (Bacher and Kendziorski 2016). Cellular sub-population structures are projected into uniform manifold approximation and projection (UMAP) plots (McInnes, Healy, and Melville 2018) and visually inspected. Putative toxin receptor candidates are identified by constructing columnar cell clusters by grouping midgut cells based on the similarity of their gene expression profiles.

### Example 6. Discovery of the Causative Mutation Gene of Cry1Fa Resistance in Columnar Cells from Brazilian Fall Armyworm

Field evolved resistance to insect control products is often receptor-mediated based on genetic linkage between highly specific receptor and toxin pairs. Recent studies showed that ATP-binding cassette (ABC) transporters serve as the major receptor family for several insect control traits based on linkage mapping of resistant strains collected from field or by cell-based validation (Banerjee et al. 2017; Flagel et al. 2018).

Among the transcripts generated from midgut using single cell sequencing described herein, sixteen short reads were assembled to a SfABCC2 allele of BR FAW population. Unexpectedly, three transcripts were mapped to the 5′ end or middle region of SfABCC2 CDS. The remaining thirteen transcripts were mapped to the C-terminus of SfABCC2 as anticipated since the dataset was generated by 3′ end Illumina NGS sequencing. The consensus sequence of the 3′ end reads is consistent with the SfABCC2 gene identified from the proprietary BR FAW transcriptome, which suggests that the midgut cell isolation, single cell encapsulation and sequencing procedures can sequence data sufficient to identify SNPs among mutated receptor genes.

### REFERENCES

Bacher, Rhonda, and Christina Kendziorski. 2016. ‘Design and computational analysis of single-cell RNA-sequencing experiments’, Genome Biology, 17:63.
Banerjee, Rahul, James Hasler, Robert Meagher, Rodney Nagoshi, Lucas Hietala, Fangneng Huang, Kenneth Narva, and Juan Luis Jurat-Fuentes. 2017. ‘Mechanism and DNA-based detection of field-evolved resistance to transgenic Bt corn in fall armyworm (Spodoptera frugiperda)’, Scientific Reports, 7:10877.
Danecek, Petr, James K Bonfield, Jennifer Liddle, John Marshall, Valeriu Ohan, Martin O Pollard, Andrew Whitwham, Thomas Keane, Shane A McCarthy, Robert M Davies, and Heng Li. 2021. ‘Twelve years of SAMtools and BCFtools’, GigaScience, 10.
Federici, Brian A. 1993. ‘Insecticidal bacterial proteins identify the midgut epithelium as a source of novel target sites for insect control’, Archives of Insect Biochemistry and Physiology, 22:357-71.
Flagel, L., Y. W. Lee, H. Wanjugi, S. Swarup, A. Brown, J. Wang, E. Kraft, J. Greenplate, J. Simmons, N. Adams, Y. Wang, S. Martinelli, J. A. Haas, A. Gowda, and G. Head. 2018. ‘Mutational disruption of the ABCC2 gene in fall armyworm, Spodoptera frugiperda, confers resistance to the Cry1Fa and Cry1A. 105 insecticidal proteins’, Sci Rep, 8:7255.
Hakim, R. S., S. Caccia, M. Loeb, and G. Smagghe. 2009. ‘Primary culture of insect midgut cells’, In Vitro Cell Dev Biol Anim, 45:106-10.
Hung, R. J., Y. Hu, R. Kirchner, Y. Liu, C. Xu, A. Comjean, S. G. Tattikota, F. Li, W. Song, S. Ho Sui, and N. Perrimon. 2020. ‘A cell atlas of the adult Drosophila midgut’, Proc Natl Acad Sci USA, 117:1514-23.
Kim, Daehwan, Joseph M. Paggi, Chanhee Park, Christopher Bennett, and Steven L. Salzberg. 2019. ‘Graph-based genome alignment and genotyping with HISAT2 and HISAT-genotype’, Nature Biotechnology, 37:907-15.
Loeb, Marcia J. 2010. ‘Factors affecting proliferation and differentiation of lepidopteran midgut stem cells’, Archives of Insect Biochemistry and Physiology, 74:1-16.
McInnes, Leland, John Healy, and James Melville. 2018. ‘Umap: Uniform manifold approximation and projection for dimension reduction’, arXiv preprint arXiv: 1802.03426.
Nagamatsu, Y., S. Toda, F. Yamaguchi, M. Ogo, M. Kogure, M. Nakamura, Y. Shibata, and T. Katsumoto. 1998. ‘Identification of Bombyx mori midgut receptor for Bacillus thuringiensis insecticidal CryIA (a) toxin’, Biosci Biotechnol Biochem, 62:718-26.
Robinson, James T., Helga Thorvaldsdóttir, Wendy Winckler, Mitchell Guttman, Eric S. Lander, Gad Getz, and Jill P. Mesirov. 2011. ‘Integrative genomics viewer’, Nature Biotechnology, 29:24-26.

All publications and published patent documents cited in this specification are incorporated herein by reference to the same extent as if each individual publication or patent document was specifically and individually indicated to be incorporated by reference.

What is claimed is:

1. A method for identifying altered expression profiles in a population of plant pests that have developed resistance to a pesticidal protein comprising:
a. providing a resistant plant pest, wherein the resistant plant pest has developed resistance to the pesticidal effects of said pesticidal protein;
b. providing a susceptible plant pest of the same species as the resistant plant pest, wherein the susceptible plant pest is susceptible to the pesticidal effects of said pesticidal protein;
c. isolating a population of cells from each of the plant pests;
d. generating a transcriptome library from each population using single cell sequencing;
e. clustering the transcripts from each library using genetic markers associated with a single cell type to generate a set of cell type specific transcripts;
f. comparing the cell type specific transcripts from resistant plant pests to the cell type specific transcripts from susceptible plant pests to identify transcripts that have an altered expression pattern in the resistant plant pest relative to the susceptible plant pest.

2. The method of claim 1, wherein said isolated population of cells consists essentially of primary midgut cells.

3. The method of claim 2, wherein the single cell type is a columnar cell, a goblet cell, or a stem cell.

4. The method of claim 3, wherein the single cell type is a columnar cell.

5. The method of claim 1, wherein the plant pest is a lepidopteran, coleopteran, hemipteran, or dipteran pest.

6. The method of claim 1, wherein a transcript having an altered expression pattern encodes a receptor protein.

7. The method of claim 6, wherein the receptor protein is selected from ATP-binding cassette (ABC) transporter, aminopeptidase N, cadherin, alkaline phosphatase, chitinase and lipase.

8. The method of claim 1, wherein the transcript having an altered expression pattern is a transcript that is an allelic variant of the same transcript in the susceptible plant pest.

9. The method of claim 1, wherein the plant pest is Plutella spp., Helicoverpa spp., Spodoptera spp., or Ostrinia spp.
